(19) United States
(12) Patent Application Publication
(10) Pub. No.: US 2010/0048540 A1
(43) Pub. Date: Feb. 25, 2010

(54) Heterocyclic N-Oxides as Hypoxic Selective Protein Kinase Inhibitors

(75) Inventors: Robert George Boyle, Cambridge (GB); Stuart Travers, Bedfordshire (GB)

Correspondence Address: HESLIN ROTHENBERG FARLEY & MESITI PC, 5 COLUMBIA CIRCLE, ALBANY, NY, 12203, US

(73) Assignee: SENTINEL ONCOLOGY LIMITED, Bedfordshire (GB)

(21) Appl. No.: 11/815,168

(22) PCT Filed: Feb. 1, 2006
(86) PCT No.: PCT/IB2006/001657

§ 371 Date: Apr. 21, 2008

(30) Foreign Application Priority Data
Feb. 1, 2005 (GB) 0501999.7

(51) Int. Cl. A61K 31/551; C07D 413/10; C07D 471/04; C07D 243/08; C07D 215/40; A61K 31/5377; A61K 31/498; A61K 31/4709; A61P 35/00

(52) U.S. Cl. 514/218; 544/118; 544/350; 540/575; 546/162; 514/234.2; 514/249; 514/313

(57) ABSTRACT

The invention relates to novel heterocyclic N-oxides which are useful as hypoxic selective cytotoxic agents that mediate and/or inhibit cell proliferation, for example, through the activity of protein kinases. The invention is further related to pharmaceutical compositions containing such compounds and compositions, and to methods of treating cancer as well as other disease states associated with unwanted ahgiogenesis and/or cellular proliferation by administering effective amounts of such compounds.

This application claims priority under 35 U.S.C §119 to UK Patent Application No. 0501999.7, filed Feb. 1, 2005.

### FIELD OF THE INVENTION

The present invention relates to heterocyclic N-oxides which are useful for inhibiting protein kinases upon in vivo selective reduction in a hypoxic environment, methods for making the compounds, their use as hypoxia selective drugs and radiosensitizers for the treatment of cancer alone or in combination with radiation and/or other anticancer agents.

### BACKGROUND OF THE INVENTION

Protein kinases constitute a large family of structurally related enzymes that are responsible for the control of a wide variety of signal transduction processes within the cell (Hardie, G. and Hanks, S. (1995) The Protein Kinase Facts Book. I and II, Academic Press, San Diego, Calif.). They do this by effecting phosphoryl transfer from a nucleoside triphosphate to a target protein that is involved in a signalling pathway. A number of these protein kinases and pathways are stimulated by extracellular stimuli, for which examples include environmental and chemical stress signals (e.g. heat shock, ultraviolet radiation, H₂O₂, osmotic shock), cytokines (e.g. interleukin-1 (IL-1) and Tumour necrosis factor α (TNF-α). Such a signal may effect several pathways within the cell and be important in the progression of many disease states. It is common that many protein kinases are unregulated or constitutively active in cancer. In addition, the cell cycle of all cells is regulated mainly by protein kinases and interference with these can cause cell death by apoptosis or programmed cell death. Protein kinases, where the up regulation leads to inappropriate proliferation include EGFR, ERBB2, PDGFR, cMet, TIE2, RET, FGFR, VEGFR, IGF-1R. Protein kinases involved in signal transduction include PKC, Akt, P70S6, PKA, PDK1, PDK2. Protein kinases involved in cell cycle progression include Cdk1, Cdk2, Cdk4, Myt1, Chk1, Wee1, AuroraA or B, Plk, Bulb1 or 3. Furthermore, protein kinases involved in response to DNA damage, include Chk1, Chk2, ATM, ATR, DNA-PK, Arg, Ab1, and CKII.

Mechanisms of cell proliferation are under active investigation at cellular and molecular levels. At the cellular level, de-regulation of signalling pathways, loss of cell cycle controls, unbridled angiogenesis or stimulation of inflammatory pathways are under scrutiny, while at the molecular level, these processes are modulated by various proteins, among which protein kinases are prominent suspects. Overall abatement of proliferation may also result from programmed cell death, or apoptosis, which is also regulated via multiple pathways, some involving proteolytic enzyme proteins.

Among candidate regulatory proteins, protein kinases are a family of enzymes that catalyze phosphorylation of the hydroxyl group of specific tyrosine, serine, or threonine residues in proteins. Typically, such phosphorylation dramatically perturbs the function of the protein, and thus protein kinases are pivotal in the regulation of a wide variety of cellular processes, including metabolism, cell proliferation, cell differentiation, and cell survival. Of the many different cellular functions in which the activity of protein kinases is known to be required, some processes represent attractive targets for therapeutic intervention for certain disease states. Two examples are cell-cycle control and angiogenesis, in which protein kinases play a pivotal role; these processes are essential for the growth of solid tumors as well as for other diseases.

Solid tumours, which make up more than 90% of all human cancers, typically have areas of very low oxygenation, or hypoxia (Brown, Molecular Medicine Today, 2000 (vol 6), 157-161). This is because the cells grow faster than the blood supply can keep up with, especially as blood flow is sluggish with very tortuous vessels, and so cells become further away from blood vessels than the diffusion distance of oxygen (100-150 μm). These hypoxic cells are resistant to killing by ionising radiation (Movsas et al., Cancer, 2000, 89, 2018; Rudat et al., Radiother. Oncol., 2000, 57, 31). Hypoxic cells are also considered to compromise response of solid tumours to cytotoxic chemotherapy (Brown and Giaccia, Cancer Res., 1998, 58, 1408). Hypoxic cancer cells also promote malignant progression and make the tumours more likely to metastasize. Typically, the more hypoxic the tumour, the harder it is to cure, a fact that has been demonstrated in many clinical trials. However, hypoxia in tumours can also be exploited and drugs have been developed to take advantage of the different chemical environment within hypoxic cancer cells. One such compound is 3-amino-1,2,4-benzotriazine 1,4-dioxide, named Tirapazamine (TPZ-Denny and Wilson, Exp Opin. Invest. Drugs, 2000, 9, 2889). Although TPZ is showing promising indications of clinical activity, at therapeutic concentrations, it also displays considerable toxicity in non-hypoxic cells giving rise to unwanted side effects such as nausea, vomiting, diarrhea, neutropenia, thrombocytopenia and muscle cramping. Given these toxic limitations, TPZ cannot be given at doses sufficient to fully exploit tumour hypoxia. There is thus a need for compounds that alone, or in combination with TPZ, exhibit enhanced hypoxic specific cytotoxicity.

It has been shown that the mono-N-oxide SR4317 can synergize with both ionising radiation and Tirapazamine by a mechanism that is proposed to be donation of the Oxygen from the mono-N-Oxide, yielding the parent heterocycle (Siim B G. Cancer Research, 2004, 64:736-742)

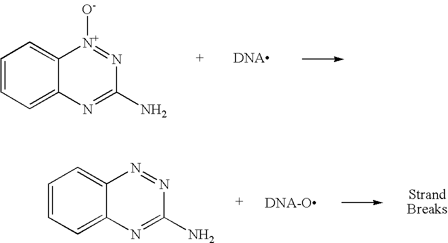

A key parameter for the successful bioreduction under hypoxia is the one-electron reduction potential, E(1). If the E(1) value is too high, reduction will not be limited to hypoxic conditions, and the compound may be toxic to normal cells. Conversely, if the E(1) value is too low, the rate of reduction may be too slow to provide therapeutic benefit. Consequently, the optimal range for hypoxic selective bioreduction appears to be between about −450 mV and −510 mV. Values higher than −300 mV have been found to induce aerobic toxicity, and values lower than −510 mV reduce slowly (Hay MP. J. Med. Chem., 2003, 46:169-182). It has been reported that mono-N-oxides of substituted 3-amino-1,2,4-benzotriazine 1,4-dioxides have E(1) values in the range required for hypoxic bioreduction, and that these values change in line with the substitution patterns (Anderson R F. Org. Biomol. Chem., 2005, 3:2167-2174)

It is an object of the present invention to provide compounds that satisfy this need for enhanced hypoxic specific toxicity. In particular, it is an object of the invention to provide N-oxides capable of selective reduction in a hypoxic tumor environment to become potent protein kinase inhibitors. Further, it is an object to provide compounds that inflict oxidative damage to DNA during their reduction to increase tumor toxicity when administered alone, and/or which potentiate the damage to tumor DNA caused by radiation treatment or drugs such as TPZ when used in combination therewith. It is expected that the compounds of the invention will have little or no protein kinase activity until selectively reduced in the hypoxic environment of a tumor. Such a mechanism will provide a safer protein kinase inhibitor and, in addition, significantly potentiate the initial DNA damaging effect of TPZ when administered together.

Accordingly, it is an object of the present invention to provide a range of heterocyclic N-oxides that are void of kinase activity in their oxidized state, but have E(1) values in the range of −300 mV to −550 mV, preferably −400 mV to −510 mV, and more preferably −450 mV to −510 mV such that they are selectively reduced under tumor hypoxia to release an active kinase inhibitor. In a second aspect of this invention, it is expected that, when administered in combination with ionising radiation or Tirapazamine or a similar DNA damaging chemotherapeutic (e.g. bleomycin) in a hypoxic environment, these molecules will potentiate the effect of the radiation or chemotherapeutic by: (a). providing an oxygen source to ‘fix’ or make permanent the DNA damage and (b) release an active kinase inhibitor, that will enhance the overall cell killing effect.

### SUMMARY OF THE INVENTION

The present invention is directed to compounds and methods for treating cancer indications through kinase inhibition and/or DNA oxidative damage. The compounds of the invention undergo selective reduction in hypoxic tumor environments to form potent inhibitors of kinases, such as Ab1, Arg, Aurora, CDKs, VEGF, and CHK-1, or cyclin complexes thereof. Further, in connection with their hypoxia induced reduction, the compounds of the invention possess, in select situations, the potential to impart oxidative damage to surrounding DNA. This additional functionality may alone provide tumor toxicity, or it may provide synergistic potentiation of the cytotoxic effect of other therapeutic treatments, such as ionizing radiation or chemotherapeutic agents such as TPZ, in hypoxic tumor cells. Accordingly, in one embodiment, the invention is directed to a method of selectively modulating or inhibiting the activity of protein kinases in hypoxic tumor cells. In another embodiment, the invention is directed to a method of selectively causing oxidative damage to DNA in hypoxic tumor cells.

In another embodiment, the invention is directed to certain heterocylic, mono-N-oxides of formulas (I), (II) and (III) having one electron reduction potential sufficient to selectively undergo reduction in a hypoxic tumor environment at sufficient rates to have therapeutic effect:

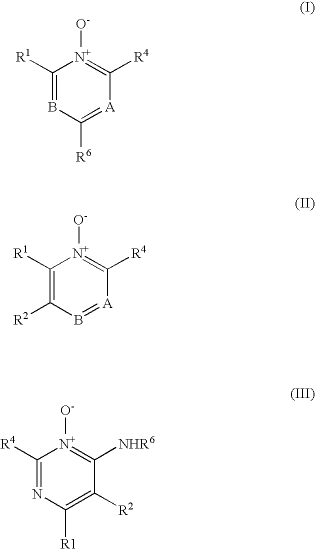

### Wherein:

R¹ and R² are each independently selected from hydrogen, C₁-C₆ alkyl which is unsubstituted or substituted, C₁-C₆ haloalkyl, C₃-C₁₀ cycloalkyl which is unsubstituted or substituted, aryl which is unsubstituted or substituted, a 5- to 7-membered heterocyclic ring which is saturated or unsaturated and which may contain one or more heteroatoms selected from O, N and S and which is unsubstituted or substituted on any ring carbon or ring heteroatom, C₁-C₆ alkoxy which is unsubstituted or substituted, C₃-C₁₀ cycloalkoxy which is unsubstituted or substituted, halogen, hydroxyl, —OR⁶, —SR⁶, —SO₂R⁶, —SO₂N(R⁶)₂, —SO₂N(R⁷)(R⁸), —N(R⁶)₂, —N(R⁷)(R⁸), cyano, nitro, —COOR⁶, —C(O)N(R⁶)₂, —C(O)N(R⁷)(R⁸), —N(R⁶)C(O)R⁶, —N(R⁶)COOR⁶, —N(R⁶)CON(R⁶)₂, —N(R⁶)CON(R⁹)(R⁸), —N(R⁶)SO(R⁶), —N(R⁶)SO₂(R⁶), —C(O)R⁶, —OCH₂(CH₂)ₚN(R⁶)₂, —OCH₂(CH₂)ₚN(R⁷)(R⁸), —CH₂(CH₂)ₚN(R⁶)₂, —CH₂(CH₂)ₚN(R⁷)(R⁸), C(O)NHCH₂(CH₂)ₚN(R⁶)₂, —C(O)NHCH₂(CH₂)ₚN(R⁷)(R⁸), —NH(CH₂)ₚN(R⁶)₂, —NH(CH₂)ₚN(R⁷)(R⁸), —NHC(O)CH₂(CH₂)ₚN(R⁷)(R⁸), —NHC(O)CH₂(CH₂)ₚN(R⁶)₂, —(CH₂)_{q}C(O)R⁶, —OCH₂CH₂OR⁶, —O(CH₂)_{q}C(O)R⁶, —O(CH₂)_{q}(OCH₂CH₂)_{q}OR⁶, XN(R⁶)₂ or —X—N(R⁷)(R⁸)

wherein X is a C₁-C₆ alkylidine group that is optionally interrupted by —O—, —S—, —C(O)— or —N(R⁶), and wherein;

R¹ and R² may form, together with the carbon atoms to which they are attached, a fused benzene ring or a fused 5- to 7-membered heterocyclic ring which is saturated or unsaturated and which may contain one or more heteroatoms selected from O, N, and S, the benzene ring or heterocyclic ring being unsubstituted or substituted;

wherein R⁶ is H, C₁-C₆ alkyl which is unsubstituted or substituted, C₃-C₁₀ cycloalkyl which is unsubstituted or substituted, a 5- to 7-membered heterocyclic ring which is unsaturated or saturated and which contains one or more heteroatoms selected from O, N and S and which is unsubstituted or substituted on any ring carbon or ring heteroatom, an aromatic or heteroaromatic ring optionally substituted by halogen, hydroxyl, —OR¹⁰, —SR¹⁰, —SO₂R¹⁰, —SO₂N(R¹⁰)₂, —N(R¹⁰)₂, —N(R⁷)(R⁸), cyano, nitro, —COOR¹⁰, —C(O)N(R¹⁰)₂, —N(R¹⁰)C(O)R¹⁰, —N(R¹⁰)COOR¹⁰, —N(R¹⁰)CON(R¹⁰)₂, —N(R¹⁰)SO(R¹⁰), —N(R¹⁰)SO₂(R¹⁰), —C(O)R¹⁰ and aromatic or heteroaromatic ring optionally substituted by two R¹⁰ that may be taken together to form a fused bicyclic system, and wherein more than one R⁶ attached to the same nitrogen atom is the same or different;

R⁷ and R⁸ form, together with the N atom to which they are attached, a 3- to 9-membered N-containing heterocyclic ring which is unsaturated or saturated and which may contain one or more heteroatoms selected from O, N and S and which is unsubstituted or substituted on any ring carbon or ring heteroatom;

p is 0 or an integer from 1 to 5;

q is an integer from 1 to 6;

A and B are optionally and independently N or CR³

wherein R³ is optionally H, NHR⁶, OR⁶, SR⁶, or selected from C₁-C₆ alkyl which is unsubstituted or substituted, C₁-C₆ alkoxy which is unsubstituted or substituted, C₃-C₁₀ cycloalkoxy which is unsubstituted or substituted, phenyl which is unsubstituted or substituted, halogen, hydroxyl, SOR⁶, SO₂R⁶, SONHR⁶, NO₂, cyano, N(R⁶)₂, NHCON(R⁶)₂ or NHCON(R⁷)(R⁸), COOR₆, NR⁷R⁸ wherein each R⁶ is the same or different and wherein R³ groups on adjacent carbon atoms can, together with the carbon atoms to which they are attached, form an aromatic ring which may be substituted with one or more R⁶ groups;

R⁴ is optionally H, NHR⁶, SR⁶, C₁-C₆ alkyl which is unsubstituted or substituted and which is optionally interrupted by —O—, —S—, —C(O)— or —N(R⁶)—, C₃-C₈ cycloalkyl which is unsubstituted or substituted, aryl which is unsubstituted or substituted or a 5- to 7-membered heterocyclic group which is unsaturated or saturated, which contains 1 or more heteroatoms selected from O, N and S and which is unsubstituted or substituted on any ring carbon or ring heteroatom, or R⁴ and A, together with the C atoms to which they are attached, form a 5-membered N-containing heterocyclic ring, which is saturated or unsaturated and which may contain one or more heteroatoms selected from O, N and S and which is unsubstituted or substituted on any ring carbon or ring heteroatom; and

R¹⁰ is H or C₁-C₆ alkyl which is unsubstituted or substituted, C₃-C₁₀ cycloalkyl which is unsubstituted or substituted or a 5- to 7-membered heterocyclic ring which is unsaturated or saturated which contains one or more heteroatoms selected from O, N and S and which is unsubstituted or substituted on any ring carbon or ring heteroatom and wherein more than one R¹⁰ attached to the same nitrogen atom is the same or different;

or a pharmaceutically acceptable salt of a compound of the Formulas (I), (II) or In particularly preferred embodiments, the present invention is directed to novel heterocyclic N-monoxides of formulas (I), (II) or (III) having one electron reduction potential (E(1)) values less than about −300 mV, and preferably in the range of about −400 mV to about −510 mV, more preferably −450 mV to −510 mV, which are useful as hypoxic selective prodrugs for cytotoxic metabolites that mediate and/or inhibit cell proliferation; for example, through the activity of protein kinases. The preferred compounds of the invention will undergo selective reduction in vivo, under hypoxic conditions, to produce the corresponding N-heterocycle active metabolite, which mediates or inhibits kinase activity.

An important requirement for binding to protein kinases is the geometry of the active molecules. The adenine moiety of ATP binds to the kinase active site by hydrogen bonding to a series of backbone amides, a feature known as hinge binding and is a common and important feature of many protein kinase inhibitors (Williams, D. H. Current opinion in Pharmacology, 2002, 2, 567-573. Accordingly, in a preferred embodiment, the compounds of the invention bind to the kinase active site via a comparable hinge binding motif. For example, in one preferred embodiment, once reduced, the azo group previously bearing the mono-oxide moiety bonds to the protein kinase such that it forms part of the hinge binding moiety between the inhibitor and the protein kinase, which renders the kinase incapable of interacting with its natural substrate. In another preferred embodiment, once reduced, the azo group previously bearing the monoxide moiety forms part of an internal H-bond that alters the compound's structural geometry into a conformation favorable for interaction with the protein kinase to render the kinase incapable of interacting with its natural substrate.

Further, in an additional embodiment of the invention, the oxidising radical liberated during reduction of the heterocylic N-monoxide prodrug may impart, or potentiate, oxidative damage to the DNA of the tumor cells. Accordingly, this invention further relates to heterocyclic N-monoxides having a one electron reduction potential too low to independently cause oxidative damage to tumor DNA in an hypoxic environment, e.g., lower than −510 mV, but that can potentiate the cytotoxic effects of Tirapazamine (TPZ) and/or ionizing radiation, as well as provide active metabolites that have protein kinase inhibitory or modulating effect. In a preferred embodiment, the heterocyclic N-monoxides having a one electron reduction potential lower than about −300 mV is derivatized from known protein kinase templates such as quinazolines, quinolines, isoquinolines, azaindoles, 7H-Pyrrolo[2,3-d]pyrimidines, 5H-Pyrrolo[2,3-b]pyrazines, pyrazines, and pyridines.

This invention further relates to pharmaceutical compositions containing compounds of the present invention, and to methods of treating cancer as well as other disease states associated with unwanted angiogenesis and/or cellular proliferation, by administering effective amounts of such compounds.

### DETAILED DESCRIPTION OF PREFERRED EMBODIMENTS OF THE INVENTION

To achieve the afore-mentioned objectives, and in accordance with the purpose of the invention, as embodied and broadly described, one aspect of the invention provides heterocyclic mono-N-oxides, that undergo selective reduction in hypoxic environments, according to formulas (I), (II) or (III):

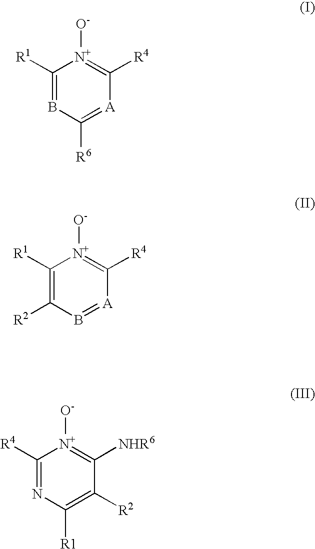

### Wherein:

R¹ and R² are each independently selected from hydrogen, C₁-C₆ alkyl which is unsubstituted or substituted, C₁-C₆ haloalkyl, C₃-C₁₀ cycloalkyl which is unsubstituted or substituted, aryl which is unsubstituted or substituted, a 5- to 7-membered heterocyclic ring which is saturated or unsaturated and which may contain one or more heteroatoms selected from O, N and S and which is unsubstituted or substituted on any ring carbon or ring heteroatom, C₁-C₆ alkoxy which is unsubstituted or substituted, C₃-C₁₀ cycloalkoxy which is unsubstituted or substituted, halogen, hydroxyl, —OR⁶, —SR⁶, —SO₂R⁶, —SO₂N(R⁶)₂, —SO₂N(R⁷)(R⁸), —N(R⁶)₂, —N(R⁷)(R⁸), cyano, nitro, —COOR⁶, —C(O)N(R⁶)₂, —C(O)N(R⁷)(R⁸), —N(R⁶)C(O)R⁶, —N(R⁶)COOR⁶, —N(R⁶)CON(R⁶)₂, —N(R⁶)CON(R⁹)(R⁸), —N(R⁶)SO(R⁶), —N(R⁶)SO₂(R⁶), —C(O)R⁶, —OCH₂(CH₂)ₚN(R⁶)₂, —OCH₂(CH₂)ₚN(R⁷)(R⁸), —CH₂(CH₂)ₚN(R⁶)₂, —CH₂(CH₂)ₚN(R⁷)(R⁸), C(O)NHCH₂(CH₂)ₚN(R⁶)₂, —C(O)NHCH₂(CH₂)ₚN(R⁷)(R⁸), —NH(CH₂)ₚN(R⁶)₂, —NH(CH₂)ₚN(R⁷)(R⁸), —NHC(O)CH₂(CH₂)ₚN(R⁷)(R⁸), —NHC(O)CH₂(CH₂)ₚN(R⁶)₂, —(CH₂)_{q}C(O)R⁶, —OCH₂CH₂OR⁶, —O(CH₂)_{q}C(O)R⁶, —O(CH₂)_{q}(OCH₂CH₂)_{q}OR⁶, XN 6)₂ or —X—N(R⁷)(R⁸)

wherein X is a C₁-C₆ alkylidine group that is optionally interrupted by —O—, —S—, —C(O)— or —N(R⁶), and wherein

R¹ and R² may form, together with the carbon atoms to which they are attached, a fused benzene ring or a fused 5- to 7-membered heterocyclic ring which is saturated or unsaturated and which may contain one or more heteroatoms selected from O, N, and S, the benzene ring or heterocyclic ring being unsubstituted or substituted;

wherein R⁶ is H, C₁-C₆ alkyl which is unsubstituted or substituted, C₃-C₁₀ cycloalkyl which is unsubstituted or substituted, a 5- to 7-membered heterocyclic ring which is unsaturated or saturated and which contains one or more heteroatoms selected from O, N and S and which is unsubstituted or substituted on any ring carbon or ring heteroatom, an aromatic or heteroaromatic ring optionally substituted by halogen, hydroxyl, —OR¹⁰, —SR¹⁰, —SO₂R¹⁰, —SO₂N(R¹⁰)₂, —N(R¹⁰)₂, —N(R⁷)(R⁸), cyano, nitro, —COOR¹⁰, —C(O)N(R¹⁰)₂, —N(R¹⁰)C(O)R¹⁰, —N(R¹⁰)COOR¹⁰, —N(R¹⁰)CON(R¹⁰)₂, —N(R¹⁰)SO(R¹⁰), —N(R¹⁰)SO₂(R¹⁰), —C(O)R¹⁰ and aromatic or heteroaromatic ring optionally substituted by two R¹⁰ that may be taken together to form a fused bicyclic system, and wherein more than one R⁶ attached to the same nitrogen atom is the same or different;

R⁷ and R⁸ form, together with the N atom to which they are attached, a 3- to 9-membered N-containing heterocyclic ring which is unsaturated or saturated and which may contain one or more heteroatoms selected from O, N and S and which is unsubstituted or substituted on any ring carbon or ring heteroatom;

p is 0 or an integer from 1 to 5;

q is an integer from 1 to 6;

A and B are optionally and independently N or CR³

wherein R³ is optionally H, NHR⁶, OR⁶, SR⁶, or selected from C₁-C₆ alkyl which is unsubstituted or substituted, C₁-C₆ alkoxy which is unsubstituted or substituted, C₃-C₁₀ cycloalkoxy which is unsubstituted or substituted, phenyl which is unsubstituted or substituted, halogen, hydroxyl, SOR⁶, SO₂R⁶, SONHR⁶, NO₂, cyano, N(R⁶)₂, NHCON(R⁶)₂ or NHCON(R⁷)(R⁸), COOR₆, NR⁷R⁸ wherein each R⁶ is the same or different and wherein R³ groups on adjacent carbon atoms can, together with the carbon atoms to which they are attached, form an aromatic ring which may be substituted with one or more R⁶ groups;

R⁴ is optionally H, NHR⁶, SR⁶, C₁-C₆ alkyl which is unsubstituted or substituted and which is optionally interrupted by —O—, —S—, —C(O)— or —N(R⁶)—, C₃-C₈ cycloalkyl which is unsubstituted or substituted, aryl which is unsubstituted or substituted or a 5- to 7-membered heterocyclic group which is unsaturated or saturated, which contains 1 or more heteroatoms selected from O, N and S and which is unsubstituted or substituted on any ring carbon or ring heteroatom, or R⁴ and A, together with the C atoms to which they are attached, form a 5-membered N-containing heterocyclic ring, which is saturated or unsaturated and which may contain one or more heteroatoms selected from O, N and S and which is unsubstituted or substituted on any ring carbon or ring heteroatom; and

R¹⁰ is H or C₁-C₆ alkyl which is unsubstituted or substituted, C₃-C₁₀ cycloalkyl which is unsubstituted or substituted or a 5- to 7-membered heterocyclic ring which is unsaturated or saturated which contains one or more heteroatoms selected from O, N and S and which is unsubstituted or substituted on any ring carbon or ring heteroatom and wherein more than one R¹⁰ attached to the same nitrogen atom is the same or different;

or a pharmaceutically acceptable salt of a compound of the Formulas (I), (II) or (III).

In a preferred embodiment, the compound is a heterocyclic mono-N-oxide of the Formula II(a):

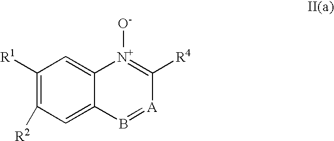

### Wherein:

R¹ and R² are each independently selected from hydrogen, C₁-C₆ alkyl which is unsubstituted or substituted, C₁-C₆ haloalkyl, C₃-C₁₀ cycloalkyl which is unsubstituted or substituted, aryl which is unsubstituted or substituted, a 5- to 7-membered heterocyclic ring which is saturated or unsaturated and which may contain one or more heteroatoms selected from O, N and S and which is unsubstituted or substituted on any ring carbon or ring heteroatom, C₁-C₆ alkoxy which is unsubstituted or substituted, C₃-C₁₀ cycloalkoxy which is unsubstituted or substituted, halogen, hydroxyl, —OR⁶, —SR⁶, —SO₂R⁶, —SO₂N(R⁶)₂, —SO₂N(R⁷)(R⁸), —N(R⁶)₂, —N(R⁷)(R⁸), cyano, nitro, —COOR⁶, —C(O)N(R⁶)₂, —C(O)N(R⁷)(R⁸), —N(R⁶)C(O)R⁶, —N(R⁶)COOR⁶, —N(R⁶)CON(R⁶)₂, —N(R⁶)CON(R⁹)(R⁸), —N(R⁶)SO(R⁶), —N(R⁶)SO₂(R⁶), —C(O)R⁶, —OCH₂(CH₂)ₚN(R⁶)₂, —OCH₂(CH₂)ₚN(R⁷)(R⁸), —CH₂(CH₂)ₚN(R⁶)₂, —CH₂(CH₂)ₚN(R⁷)(R⁸), C(O)NHCH₂(CH₂)ₚN(R⁶)₂, —C(O)NHCH₂(CH₂)ₚN(R⁷)(R⁸), —NH(CH₂)ₚN(R⁶)₂, —NH(CH₂)ₚN(R⁷)(R⁸), —NHC(O)CH₂(CH₂)ₚN(R⁷)(R⁸), —NHC(O)CH₂(CH₂)ₚN(R⁶)₂, —(CH₂)_{q}C(O)R⁶, —OCH₂CH₂OR⁶, —O(CH₂)_{q}C(O)R⁶, —O(CH₂)_{q}(OCH₂CH₂)_{q}OR⁶, XN(R⁶)₂ or —X—N(R⁷)(R⁸)

wherein X is a C₁-C₆ alkylidine group that is optionally interrupted by —O—, —S—, —C(O)— or —N(R⁶), and

wherein R⁶ is H, C₁-C₆ alkyl which is unsubstituted or substituted, C₃-C₁₀ cycloalkyl which is unsubstituted or substituted, a 5- to 7-membered heterocyclic ring which is unsaturated or saturated and which contains one or more heteroatoms selected from O, N and S and which is unsubstituted or substituted on any ring carbon or ring heteroatom, an aromatic or heteroaromatic ring optionally substituted by halogen, hydroxyl, —OR¹⁰, —SR¹⁰, —SO₂R¹⁰, —SO₂N(R¹⁰)₂, —N(R¹⁰)₂, —N(R⁷)(R⁸), cyano, nitro, —COOR¹⁰, —C(O)N(R¹⁰)₂, —N(R¹⁰)C(O)R¹⁰, —N(R¹⁰)COOR¹⁰, —N(R¹⁰)CON(R¹⁰)₂, —N(R¹⁰)SO(R¹⁰), —N(R¹⁰)SO₂(O), —C(O)R¹⁰ and aromatic or heteroaromatic ring optionally substituted by two R¹⁰ that may be taken together to form a fused bicyclic system, and wherein more than one R⁶ attached to the same nitrogen atom is the same or different;

R⁷ and R⁸ form, together with the N atom to which they are attached, a 3- to 9-membered N-containing heterocyclic ring which is unsaturated or saturated and which may contain one or more heteroatoms selected from O, N and S and which is unsubstituted or substituted on any ring carbon or ring heteroatom;

p is 0 or an integer from 1 to 5;

q is an integer from 1 to 6;

A and B are optionally and independently N or CR³

wherein R³ is optionally H, NHR⁶, OR⁶, SR⁶, or selected from C₁-C₆ alkyl which is unsubstituted or substituted, C₁-C₆ alkoxy which is unsubstituted or substituted, C₃-C₁₀ cycloalkoxy which is unsubstituted or substituted, phenyl which is unsubstituted or substituted, halogen, hydroxyl, SOR⁶, SO₂R⁶, SONHR⁶, NO₂, cyano, N(R⁶)₂, NHCON(R⁶)₂ or NHCON(R⁷)(R⁸), COOR₆, NR⁷R⁸ wherein each R⁶ is the same or different and wherein R³ groups on adjacent carbon atoms can, together with the carbon atoms to which they are attached, form an aromatic ring which may be substituted with one or more R⁶ groups;

R⁴ is optionally H, NHR⁶, SR⁶, C₁-C₆ alkyl which is unsubstituted or substituted and which is optionally interrupted by —O—, —S—, —C(O)— or —N(R⁶)—, C₃-C₈ cycloalkyl which is unsubstituted or substituted, aryl which is unsubstituted or substituted or a 5- to 7-membered heterocyclic group which is unsaturated or saturated, which contains 1 or more heteroatoms selected from O, N and S and which is unsubstituted or substituted on any ring carbon or ring heteroatom, or R⁴ and A, together with the C atoms to which they are attached, form a 5-membered N-containing heterocyclic ring, which is saturated or unsaturated and which may contain one or more heteroatoms selected from O, N and S and which is unsubstituted or substituted on any ring carbon or ring heteroatom; and

R¹⁰ is H or C₁-C₆ alkyl which is unsubstituted or substituted, C₃-C₁₀ cycloalkyl which is unsubstituted or substituted or a 5- to 7-membered heterocyclic ring which is unsaturated or saturated which contains one or more heteroatoms selected from O, N and S and which is unsubstituted or substituted on any ring carbon or ring heteroatom and wherein more than one R¹⁰ attached to the same nitrogen atom is the same or different;

or a pharmaceutically acceptable salt thereof.

In another preferred embodiment, the compound is a heterocyclic mono-N-oxide of the formula II(b):

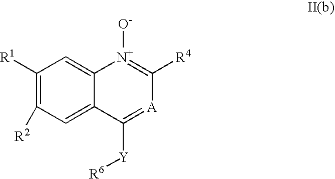

### Wherein:

Y is O, S, NH, NR⁷R⁸;

R⁴ is H or NH₂; and

R¹, R², R⁶, R⁷, R⁸ and A are as described above in formula II(a);

or a pharmaceutically acceptable salt thereof

In a further preferred embodiment, the compound is a quinazoline of the formula II(c):

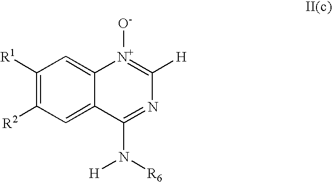

wherein R₁, R₂ and R₆ are as described above in formula II(a); or a
pharmaceutically acceptable salt thereof.

In another preferred embodiment, the compound is a quinoline of the formula II(d)

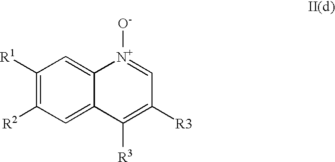

wherein R1, R2 and R3 are as described above in formula II(a); or a

pharmaceutically acceptable salt thereof.

In another preferred embodiment, the compound is a quinoxaline of the formula II(e)

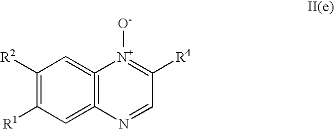

wherein R¹, R² and R⁴ are as described above in formula II(a); or a pharmaceutically acceptable salt thereof.

In another preferred embodiment, the compound is a pyrazine of the formula II(f)

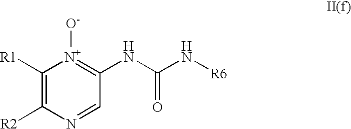

wherein R¹, R² and R⁶ are as described above in formulas I, II and III; or a
pharmaceutically acceptable salt thereof.

In another preferred embodiment, the compound is a pyridine of the formula II(g)

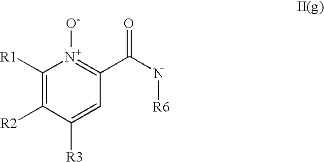

wherein R¹, R², R³ and R⁶ are as described above in formulas I, II and III; or a
pharmaceutically acceptable salt thereof.

In another preferred embodiment, the compound is an isoquinoline of the formula II(h):

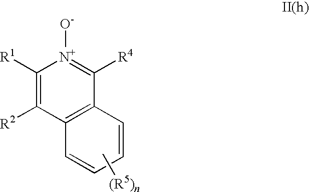

wherein R¹, R² and R⁴ are as described above in formulas I, II and III, each R⁵, which are the same or different, are as described above for R¹ and R²; and n is an integer from 1 to 4; or a pharmaceutically acceptable salt thereof.

In a further preferred embodiment, the compound is of formula I(a)

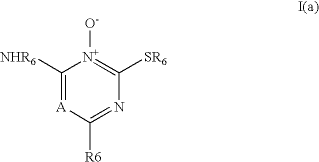

wherein A and R⁶ are as described above in formulas I, II and III; or a pharmaceutically acceptable salt thereof.

In a further preferred embodiment, the compound is a pyridine of the formula II(i)

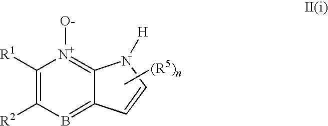

wherein B, R¹, R², and R⁵ are as described above in formulas I, II, III; and II(h); n is 1 or 2; or a pharmaceutically acceptable salt thereof.

As used in the above formulas:

A C₁-C₆ alkyl group is typically a C₁-C₄ alkyl group, for example a methyl, ethyl, n-propyl, i-propyl, n-butyl, sec-butyl or tert-butyl group. A C₁-C₆ alkyl group is unsubstituted or substituted, typically by one or more of the groups specified above as options for R¹. More typically a C₁-C₆ alkyl group which is unsubstituted or substituted by one or more groups selected from halogen, hydroxyl, C₁-C₆ alkoxy, nitro, amino, cyano, aryl which is unsubstituted or substituted, a 5- to 7-membered heterocyclic group as defined above (such as morpholinyl, piperidinyl, piperazinyl or pyridyl), —N(R⁶)₂, —SR⁶ and —COOR⁶ wherein R⁶ is as defined above.

A C₁-C₆ alkyl group substituted by halogen may be denoted by the term “halo-C₁-C₆ allyl”, which means an alkyl group in which one or more hydrogens is replaced by halo. A halo-C₁-C₆ alkyl group preferably contains one, two or three halo groups. A preferred example of such a group is trifluoromethyl.

A halogen is F, Cl, Br or I. Preferably it is F, Cl or Br.

A C₁-C₆ alkoxy group is linear or branched. It is typically a C₁-C₄ alkoxy group, for example a methoxy, ethoxy, propoxy, i-propoxy, n-propoxy, n-butoxy, sec-butoxy or tert-butoxy group. A C₁-C₆ alkoxy group is unsubstituted or substituted, typically by one or more groups selected from those specified above as substituents for C₁-C₆ alkyl.

A C₃-C₁₀ cycloalkyl group may be, for instance, a C₃-C₈ cycloalkyl such as cyclopropyl, cyclobutyl, cyclopentyl, cyclohexyl or cycloheptyl. Typically it is C₃-C₆ cycloalkyl. A C₃-C₁₀ cycloalkyl group is unsubstituted or substituted, typically by one or more groups selected from these specified above as substituents for C₁-C₆ alkyl.

A C₃-C₁₀ cycloalkoxy group is a group —O-cycloalkyl wherein the cycloalkyl moiety contains from 3 to 10 carbon atoms. Typically it is a C₃-C₈ or C₃-C₆ cycloalkoxy group. It may be, for instance, a cyclopropoxy, cyclobutoxy, cyclopentoxy, cyclohexoxy, cycloheptoxy or cyclooctoxy group.

An alkylidine group is a polymethylene group, i.e. —(CH₂)ₙ— wherein n is a positive integer. Preferably, n is an integer from 1 to 6.

When R¹ and R² form, together with the carbon atoms to which they are attached, a benzene ring or a 5- or 6-membered heterocyclic ring, the resulting fused bicyclic heterocycle is typically a benzotriazine, quinazoline, benzopyridazine, tetrahydrobenzotriazine, tetraliydroquinazoline, tetrahydrobenzopyridazine, pyranotriazine, dihydropyranotriazine, pyridotriazine, pyridopyrimidine, pyridopyridazine, pyrimidotriazine, pyrimidopyrimidine, pyrimidopyridazine, pyrrolotriazine, pyrrolopyrimidine, pyrrolopyridazine, oxazolotriazine, oxazoloquinoline, oxazolopyridazine, thienotriazine, thienoquinoline, thienopyridazine, furotriazine, furoquinoline, furopyridazine, thiazolotriazine, thiazoloquinoline, thiazolopyridazine, imadazotriazone, imidazoquinoline or imidazopyridazine.

A thienotriazine may be a thieno[2,3-e]triazine or a thieno[3,2-e]triazine. A pyrrolotriazine may be a pyrrolo[2,3-e]triazine or a pyrrolo[3,2-e]triazine. A furotriazine may be a furo[2,3-e]triazine or a furo[3,2-e]triazine. A thiazolotriazine may be a thiazolo[4,5-e]triazine or a thiazolo[5,4-e]triazine. An oxazolotriazine may be an oxazolo[4,5-e]triazine or an oxzazolo[5,4-e]triazine. An imidazotriazine is typically 5H-imidazo[4,5-e]triazine or 7H-imidazo[4,5-e]triazine

A 3- to 9-, or 5- to 7-membered N-containing heterocyclic ring which is unsaturated or saturated and contains 0, 1, or 2 additional heteroatoms selected from O, N, and S may be, for example, imidazolyl, imidazolinyl, imidazolidinyl, perhydropyridazyl, pyridazyl, pyridyl, pyrrolyl, pyrrolinyl, pyrrolidinyl, pyrazolyl, pyrazinyl, piperidinyl, pyrazolinyl, piperazinyl, pyrimidinyl, pyridazinyl, morpholinyl, thiamorpholinyl, triazolyl, tetrazolyl, isothiazolyl, thiazolyl, thiadiazolyl, thiazolidinyl, oxazolyl, isoxazolyl, oxadiazolyl and oxadiazolidinyl. Preferred examples of such heterocycles are pyridyl, pyrrolyl, pyrrolinyl, piperidinyl, piperazinyl and morpholinyl. The N-containing heterocycle is unsubstituted or substituted on any ring carbon or ring heteroatom, for instance by one or more groups specified above as substituents for C₁-C₆ alkyl. Preferably the substituent is one of the options defined above for R⁵.

A 5- to 7-membered heterocyclic group containing one or more heteroatoms selected from O, N and S is unsaturated or saturated. Suitable examples include those specified above as examples of a 5- to 7-membered N-containing heterocyclic ring. Further examples include furyl, thienyl, pyranyl, tetrahydropyranyl, tetrahydrofuranyl, thiazolyl and thiophenyl rings. Preferably the group is one of the above mentioned N-containing heterocyclic groups or pyrrolyl, furyl, pyridyl, piperidinyl or morpholinyl. The 5- to 7-membered heterocyclic group is unsubstituted or substituted on any ring carbon atom or ring heteroatom, for instance by one or more of the groups specified above as substituents for C₁-C₆ alkyl. Typically the substituent is halogen, C₁-C₆ alkyl or halo-C₁-C₆ alkyl.

An aryl group is a carbocyclic aromatic radical containing from 6-14 carbon atoms, preferably 6-10 atoms. Examples include phenyl, napthyl, indenyl and indanyl groups. An aryl group is unsubstituted or substituted, for instance by one or more of the groups specified above as substituents for C₁-C₆ alkyl. Preferably the substituent is one of the options specified above for R¹. Typically an aryl group is substituted by C₁-C₆ alkyl, halo-C₁-C₆ alkyl or halogen.

When R¹ or R² is —N(R⁶)₂, —OCH₂(CH₂)ₚN(R⁶)₂, —CH₂(CH₂)ₚN(R⁶)₂, —C(O)NHCH₂(CH₂)ₚN(R⁶)₂, —C(O)N(6)₂, —NH(CH₂)ₚ(R⁶)₂ or —X—N(R⁶)₂ the groups R¹ are typically, independently, H or C₁-C₆ alkyl. Alternatively, where R¹ or R² is —N(R⁷)(R⁸), —OCH₂(CH₂)ₚN(R⁷)(R⁸), —CH₂(CH₂)ₚN(R⁷)(R⁸), —C(O)NHCH₂(CH₂)ₚN(R⁷)(R⁸), —C(O)N(R⁷)(R⁸), —NH(CH₂)ₚ(R⁷)(R⁸) or -A-N(R⁷)(R⁸), R⁷ and R⁸ form an N-containing heterocyclic ring as defined above, preferably a morpholino, piperidinyl or piperazinyl group, which is unsubstituted or substituted by C₁-C₆ alkyl. Preferred examples of such heterocyclic options for R⁷ and R⁸ include piperazin-1-yl, 4-methyl-piperazin-1-yl and morpholin-4-yl groups.

There is also provided, in accordance with the invention, a pharmaceutical composition comprising a hypoxic selective prodrug of the formulas I-III, or more preferably, formulas I(a) and II(a)-II(i), which is converted into an active metabolite exhibiting inhibition of protein kinase activity when reduced in a hypoxic environment. Preferably, the mono-N-oxide moiety of the prodrug has a one electron reduction potential less than about −300 mV, more preferably in the range from about −400 mV to about −510 mV, more preferably from about −450 mV to about −510 mV. In addition to compounds of the formulas I-III, a pharmaceutically acceptable salt of such compounds may also be used. Further, as is common in the art, the compounds of the invention, including the salt forms, may be formulated into pharmaceutical preparations with conventional carriers, diluents, fillers, surfactants, and excipients known in the art.

There is further provided, in accordance with the invention, a method of using the prodrug compounds to selectively release a protein kinase modulating agent for treating a disease or disorder mediated by inhibition of kinase activity, comprising administering to a patient in need thereof, therapeutically effective amounts of a compound of Formulas I-III, or a pharmaceutically acceptable salt thereof. The method is particularly suitable for treating malignancies or cancer as well as other disease states associated with unwanted angiogenesis and/or cellular proliferation. Thus, the invention is also directed to methods of treating such diseases by administering an effective amount of the inventive agent.

Another aspect of the invention is the use of the mono-N-oxides herein described having a one electron potential lower than about −300 mV, preferably in the range of from about −400 mV to about −510 mV as radiosentizers or potentiators of oxidative DNA damage caused by chemotherapeutic agents such as Tirapazamine and/or ionizing radiation. When administered concurrently with Tirapazamine and/or ionizing radiation, the selective reduction of the mono-N-oxide prodrugs that occurs in the hypoxic environment of the tumor cells, not only releases protein kinase inhibitors, the liberated oxygen atoms have the potential to increase the effectiveness of the TPZ and/or radiation.

It is to be understood that both the foregoing general description and the following detailed description are exemplary and explanatory, and are not intended to limit the invention as claimed. Other objects and features of the invention will become apparent from the practice of the invention and the following detailed description. All references cited in this specification are expressly incorporated herein by reference.

Within the invention it is understood that a compound of Formulas I-III may exhibit the phenomenon of tautomerism, and that the formula drawings within this specification represent only one of the possible tautomeric forms. It is to be understood that the invention encompasses any tautomeric form which modulates and/or inhibits kinase activity and is not to be limited merely to any one tautomeric form utilized within the formula drawings.

Some of the inventive compounds may exist as single stereoisomers (i.e., essentially free of other stereoisomers), racemates, and/or mixtures of enantiomers and/or diastereomers. All such single stereoisomers, racemates and mixtures thereof are intended to be within the scope of the present invention. Preferably, the inventive compounds that are optically active are used in optically pure form.

As generally understood by those skilled in the art, an optically pure compound having one chiral center (i.e., one asymmetric carbon atom) is one that consists essentially of one of the two possible enantiomers (i.e., is enantiomerically pure), and an optically pure compound having more than one chiral center is one that is both diastereomerically pure and enantiomerically pure. Preferably, the compounds of the present invention are used in a form that is at least 90% optically pure, that is, a form that contains at least 90% of a single isomer (80% enantiomeric excess (“e.e.”) or diastereomeric excess (“d.e.”)), more preferably at least 95% (90% e.e. or d.e.), even more preferably at least 97.5% (95% e.e. or d.e.), and most preferably at least 99% (98% e.e. or d.e.).

Additionally, Formulas I-III are intended to cover solvated as well as unsolvated forms of the identified structures. For example, Formulas I-III include compounds of the indicated structure in both hydrated and non-hydrated forms. Other examples of solvates include the structures in combination with isopropanol, ethanol, methanol, DMSO, ethyl acetate, acetic acid, or ethanolamine.

### EXAMPLES

### Preparation of Heterocyclic Mono-N-Oxides of the Invention

### Example 1

### Quinazoline-1-Oxide

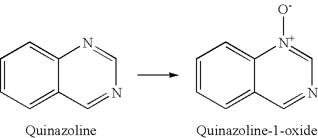

Examples of current kinase inhibitors with this scaffold includee:

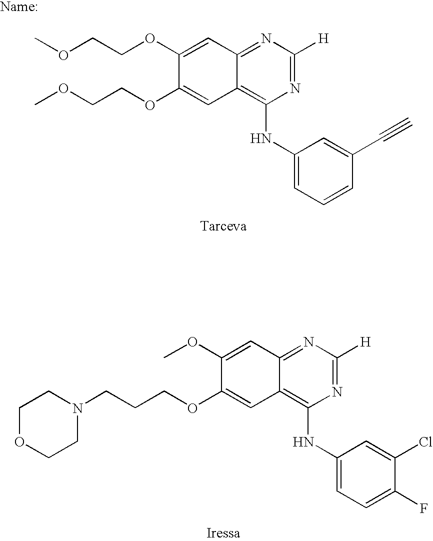

Methods for the preparation of these compounds can be found in patent documents WO9630347 and WO9633980, the disclosures of which are incorporated herein by reference. The following scheme represents the basic synthetic protocol:

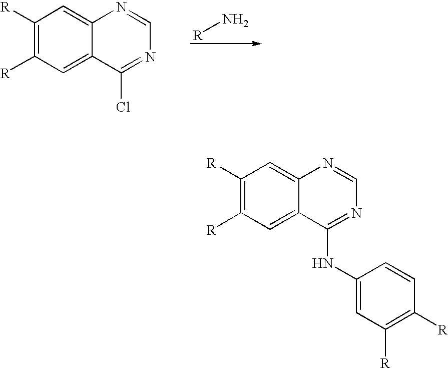

The preparation of analogous N-oxide derivatives would be carried following the protocol in patent document GB2189238 (using mCPBA for the oxidation of quinazoline derivatives to quinazoline-1-oxides). Alternatives to the use of MCPBA may be H₂O₂ (as exemplified by the oxidation of quinazolinones as disclosed in U.S. Pat. No. 4,377,408) or CF₃CO₂H as exemplified in JMC 2003, 46, 169-182. Two routes are possible as outlined below.

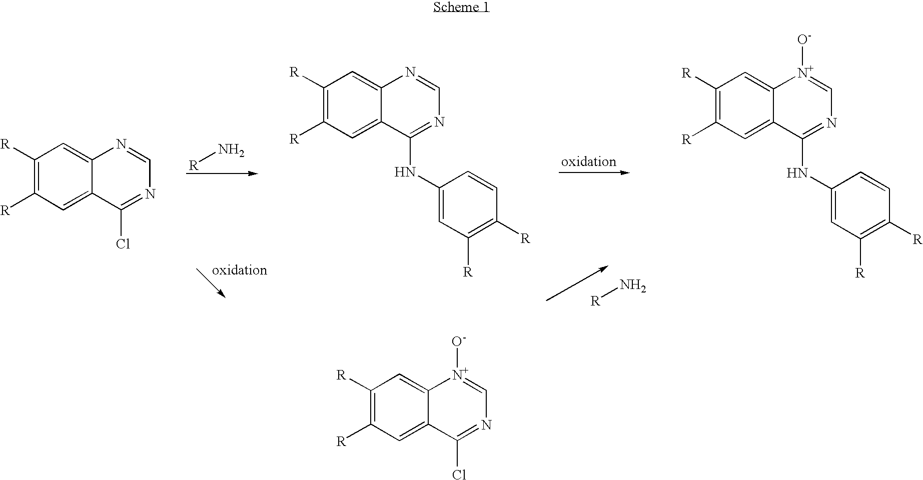

Exemplary compounds of formula II(c) include:

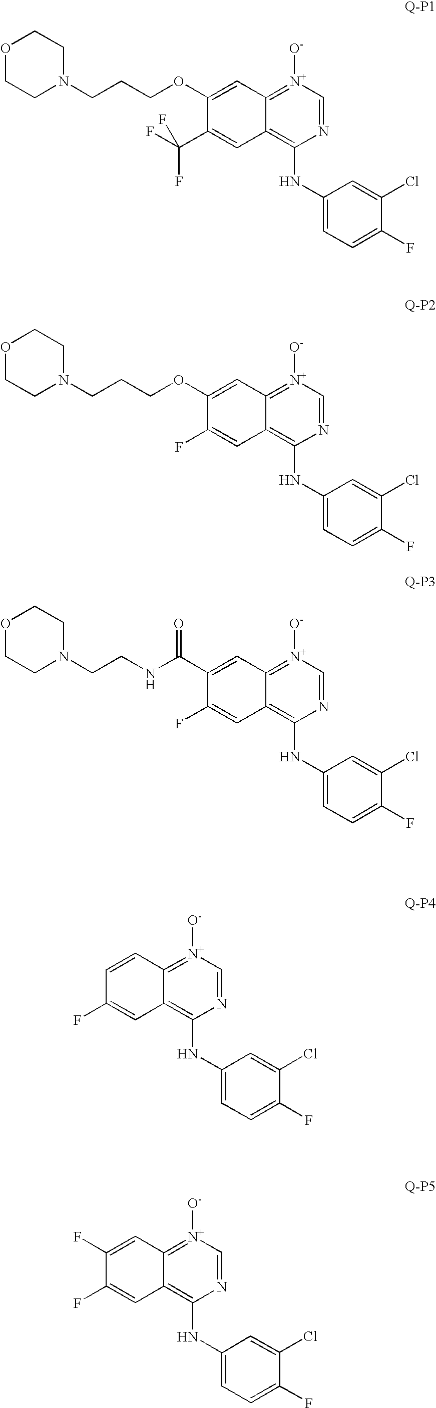

Synthesis:

P1 & P2: Protocol described in JMC 2002, 45, 3772-3793 (scheme 2).

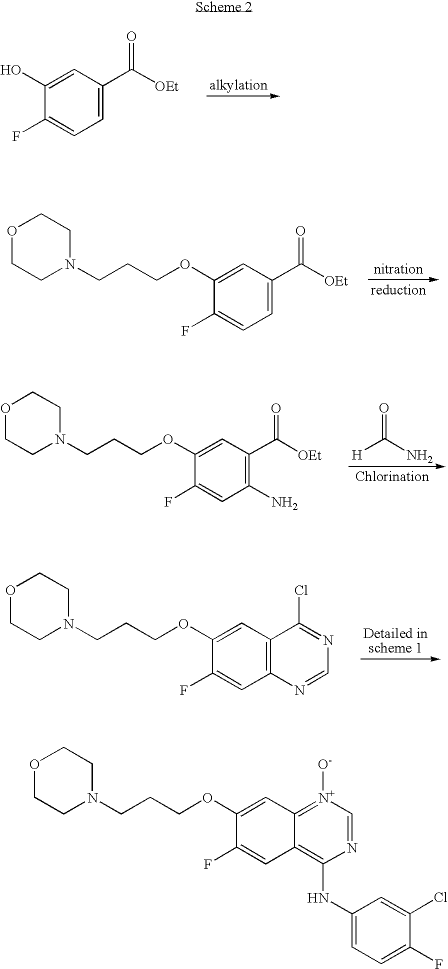

P4 & 5: Protocol described in patent document WO2005105761 (scheme 3).

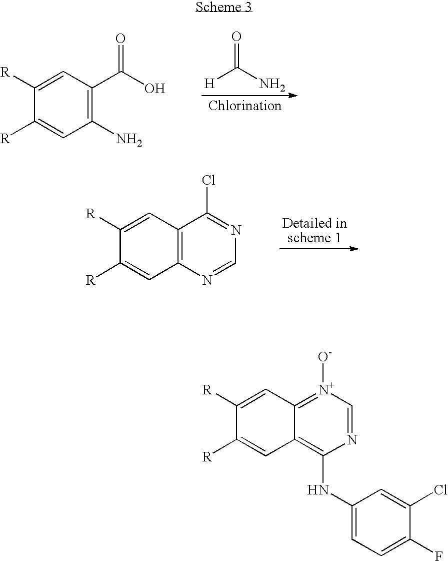

P3: Protocol described in patent document WO2005105761 (scheme 4).

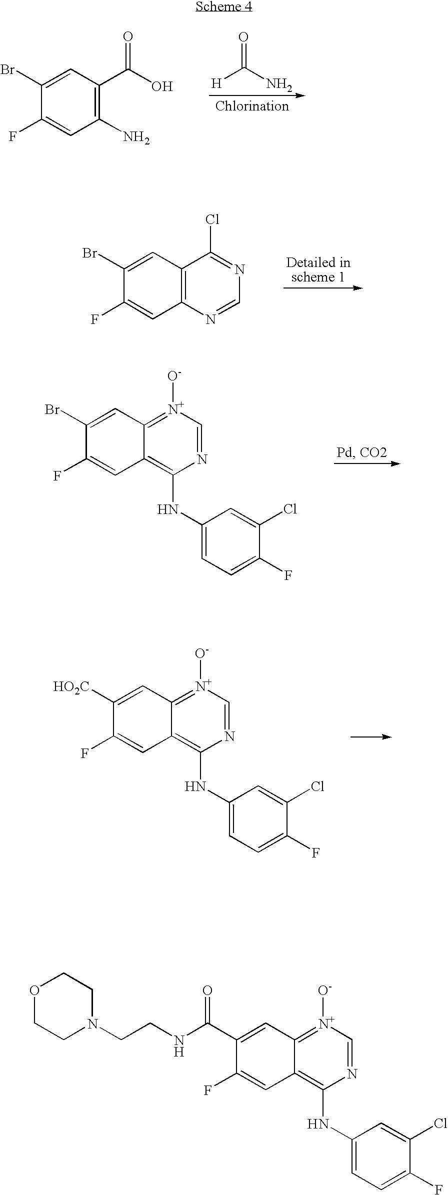

### Example 2

### Quinoxaline-1-oxide

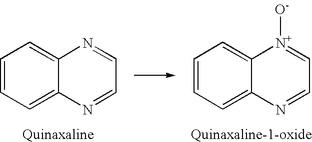

Exemplary compounds include:

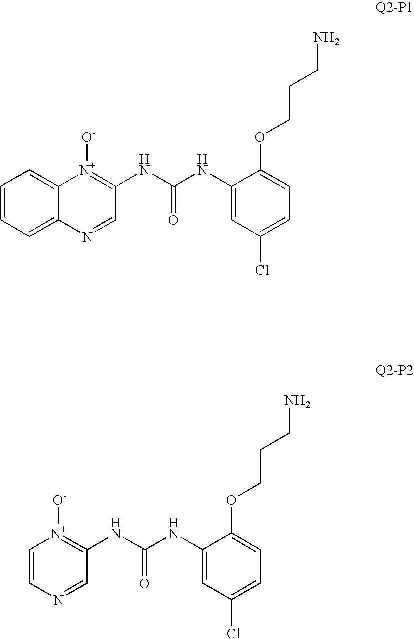

Synthesis

Q2-P1: Step A—Journal of the Chemical Society, Perkin Transactions 1: Organic and Bio-Organic Chemistry 1973, (22), 2707-13. For the synthesis of the Boc-Amino fragment see: patent document WO03101444 (Scheme 5).

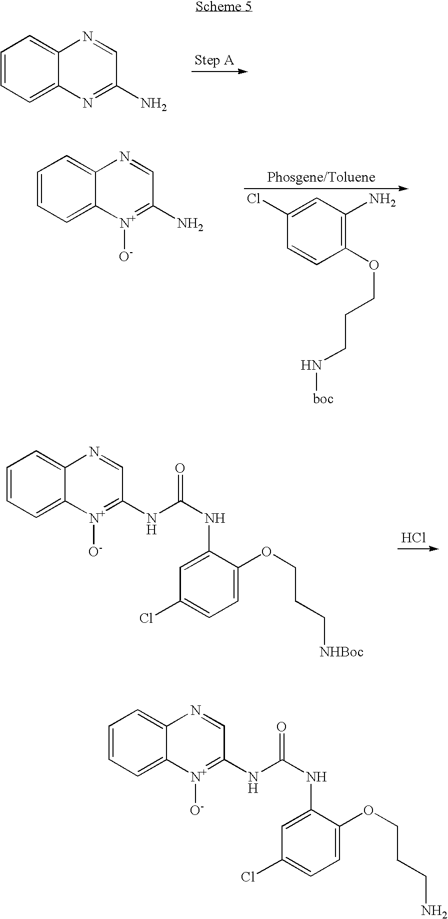

Q2-P2: The preparation of 1-Oxy-pyrazin-2-ylamine is described in Khimiya Geterotsiklicheskikh Soedinenii, 1968, 4(4), 725-8 (scheme 6). The amine would subsequently be used as described in scheme 5, above.

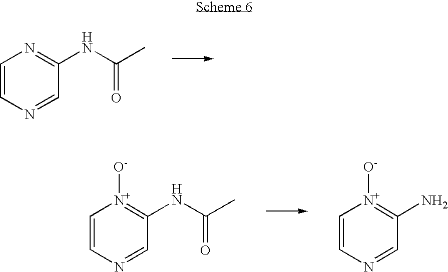

### Example 3

### Quinoline-1-Oxide

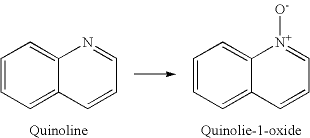

Examples of current kinase inhibitors with this scaffold include:

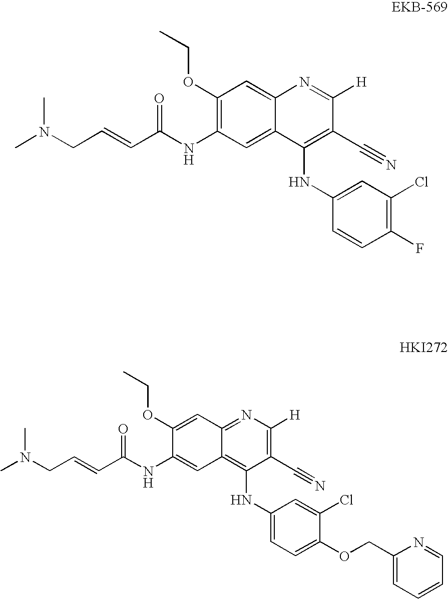

Preparation: Generic method for the preparation of parent compounds can be found in patent document WO2005070890, J. Med. Chem. 2003, 46, 49-63 and J. Med. Chem. 2005, 48, 1107-1131.

Exemplary compounds of formula II(d) include:

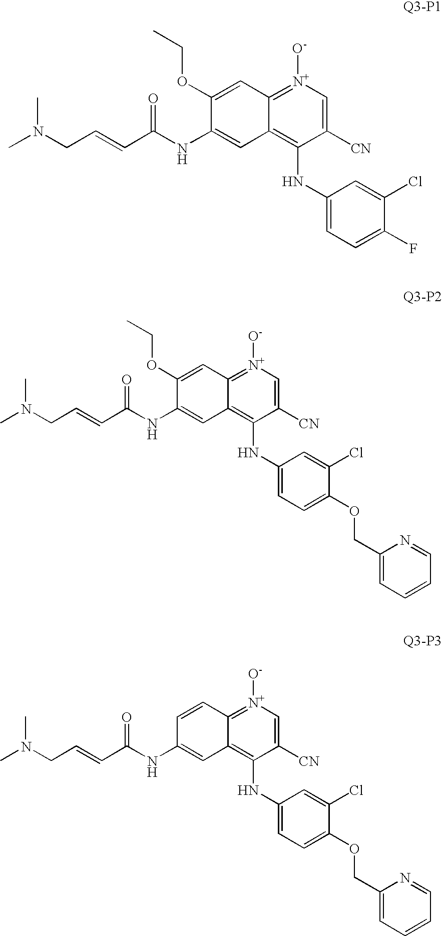

Synthesis

Q3-P1: Protocol based on the prior art publications detailed above (scheme 7)

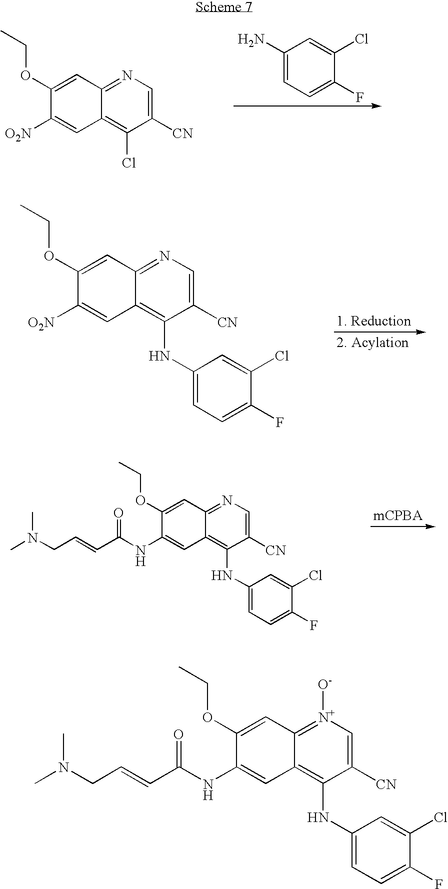

Q3-P2 (also feasible for Q3-P2, scheme 8)

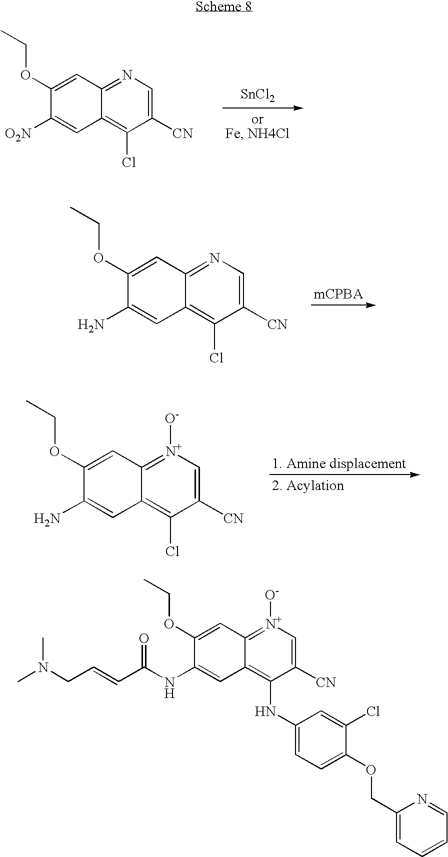

### Example 4

### Isoquinoline-2-oxide

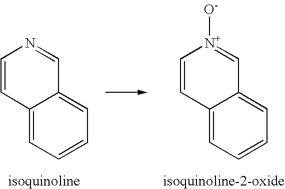

Exemplary compound of formula II(h) include:

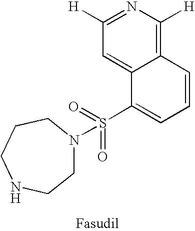

Synthesis detailed in U.S. Pat. No. 4,678,783 and the N-Oxide could be prepared by following the protocol outlined in scheme 9. For preparation of the N-Oxide by oxidation using m-CPBA, see patent document GB2189238.

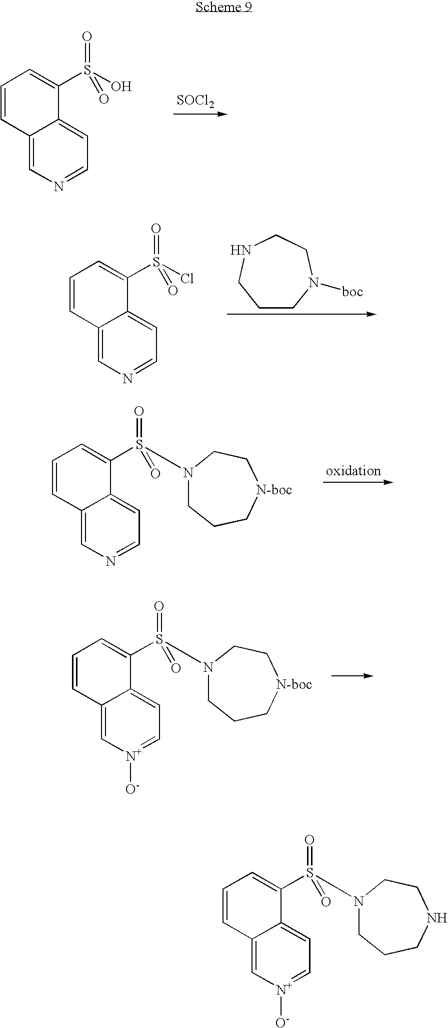

### Example 5

### 1H-Pyrrolo[2,3-b]pyridine 7-oxide

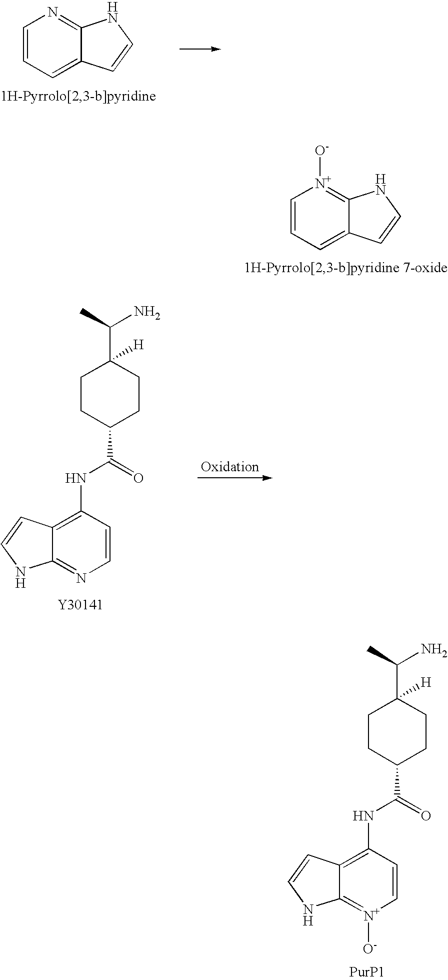

Preparation described in patent document WO9305021. Oxidation of purines and related heterocycles is described (e.g. Synlett (2001), 1, 73-74)

Exemplary compounds of formula II(i) include:

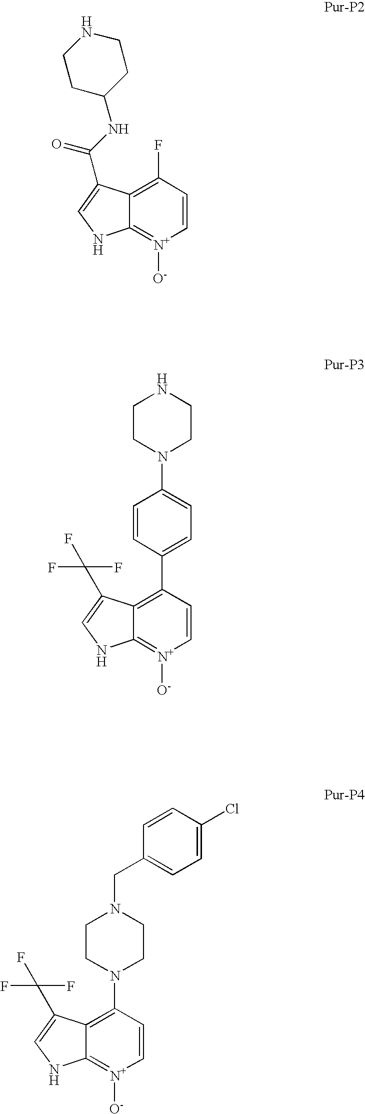

Synthesis

Pur-P2: Protocol for Step A can be found in patent document WO2005028475. The oxidation protocol is described in US Patent Application No. 2004044025 (Scheme 10).

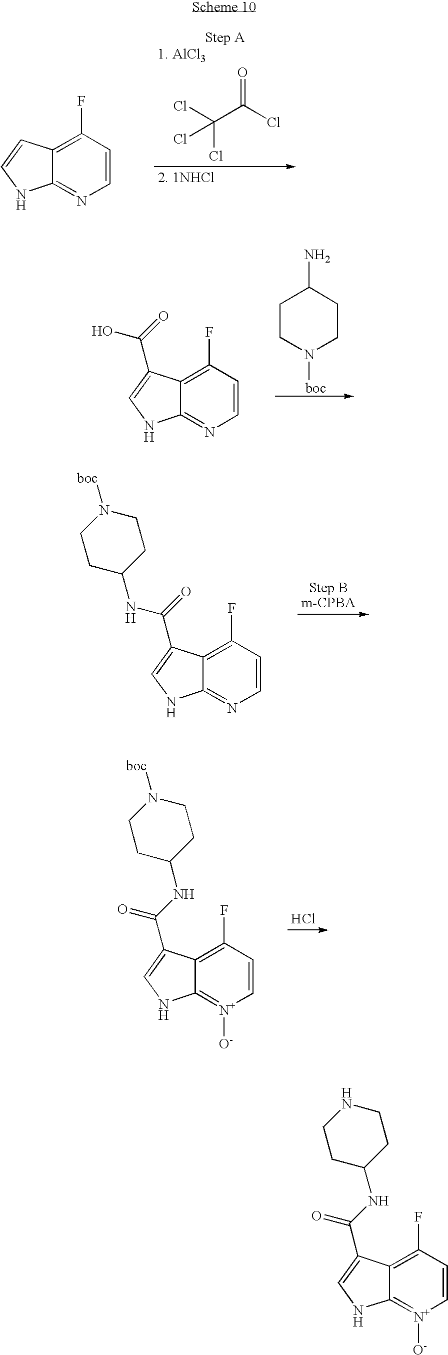

Pur-P3: Starting material 1 and the protocol for step B are described in patent document WO2005108397. Starting material 2 and the coupling protocol are described in Journal of Organic Chemistry (2003), 68(7), 2633-2638 (Scheme 11). In addition, for the coupling of aryl-chlorides using the Fu catalyst is reported to be efficient (JACS 2000, 122, 4020-4028).

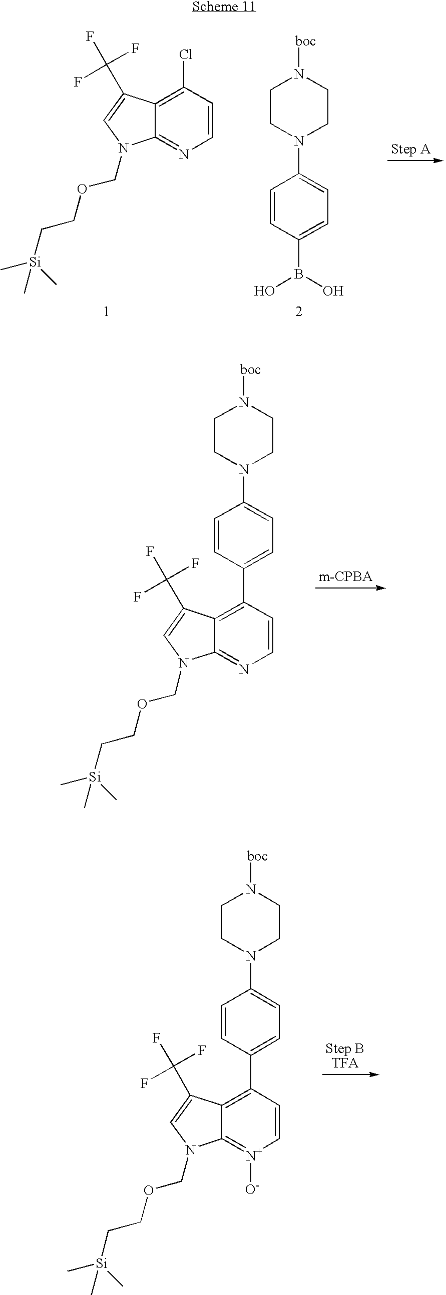

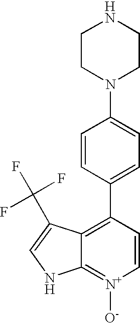

Pur-P4: Following the above scheme, however using the buchwald protocol (J. Org. Chem. 2000, 65, 1158-1174) for the amination step A (Scheme 12).

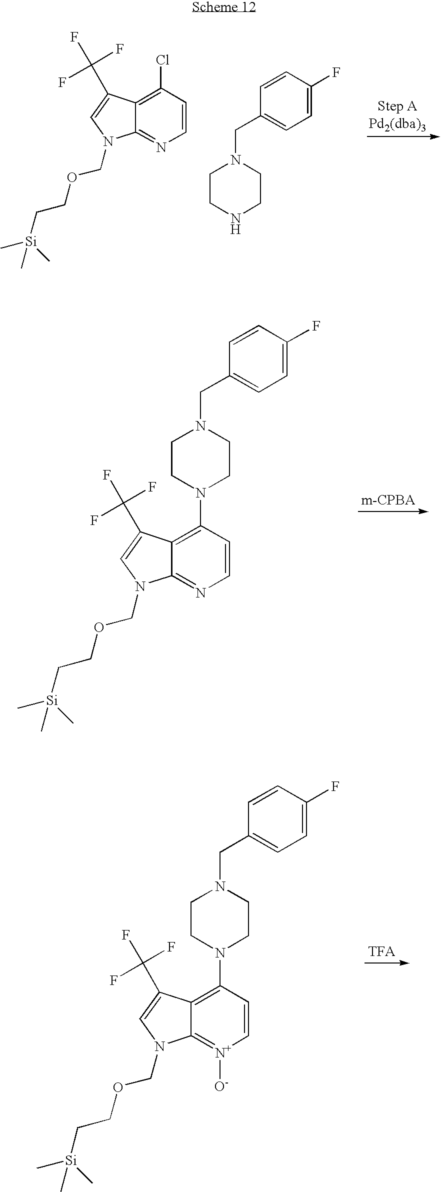

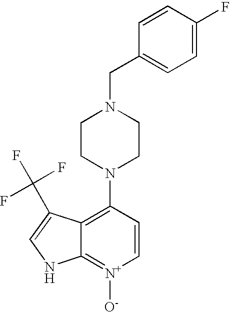

### Example 6

### 5H-Pyrrolo[2,3-b]pyrazine 4-oxide

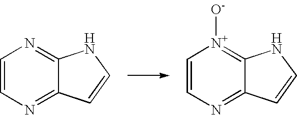

Exemplary compounds include:

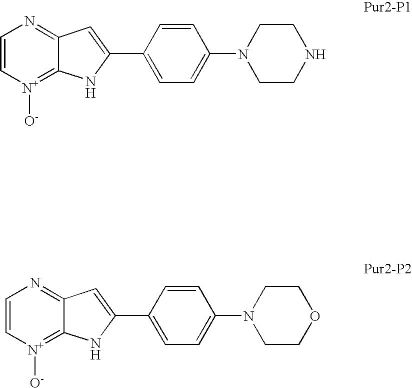

Synthesis

The protocol for step A and the synthesis of 6-(4-Bromo-phenyl)-5H-pyrrolo[2,3-b]pyrazine is described in US2005/0267304. Buchwald protocol is described in J. Org. Chem. 2000, 65, 1158-1174. The oxidation to the mono N-Oxide is not described in the literature, however it is expected that this product may be obtained by oxidation with m-CPBA (note that this oxidation is not reported and it is likely that a mixture of product N-Oxides will be formed and that the desired product would be purified by standard chromatography or HPLC).

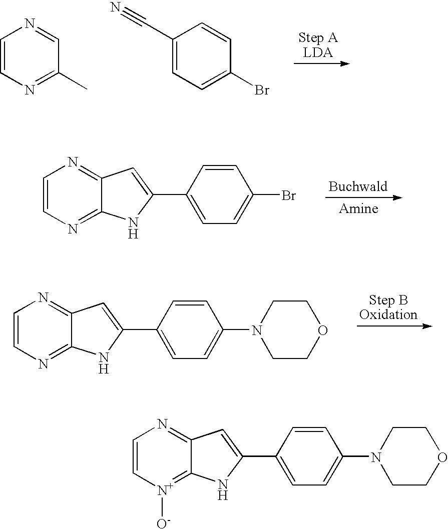

### Example 7

### Pyridine-1-oxide

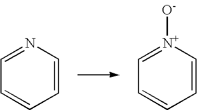

Exemplary compounds of formula II(i) include:

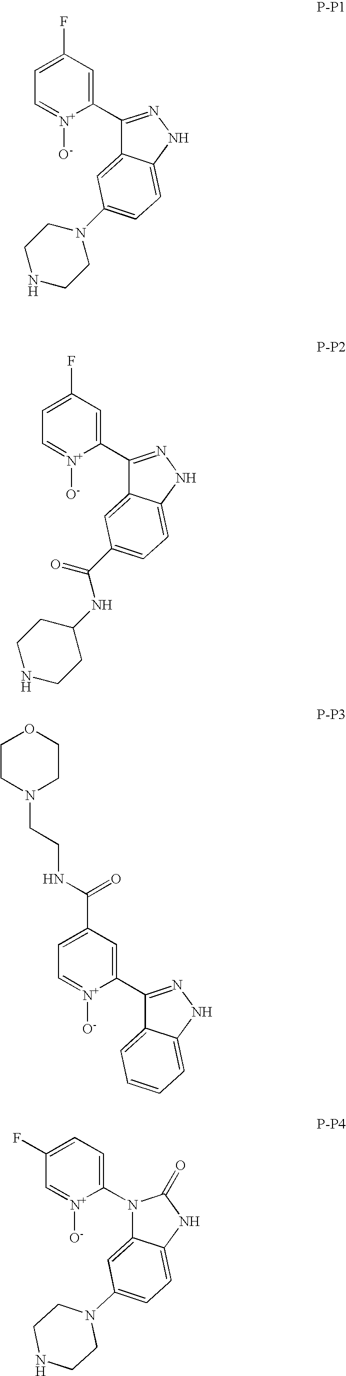

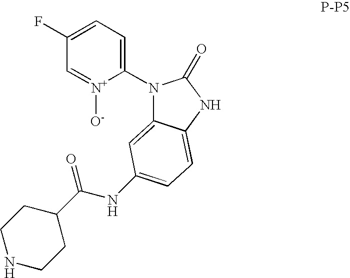

Synthesis:. 4-Fluoro-pyridine can be found in Journal of Chemical Physics (2005), 123(11), 114303/1-114303/6.

P-P1: For steps A, B, C and E the methodology is described in WO2005085227. For step D, the Buchwald procedure is described in J. Org. Chem. 2000, 65, 1158-1174.

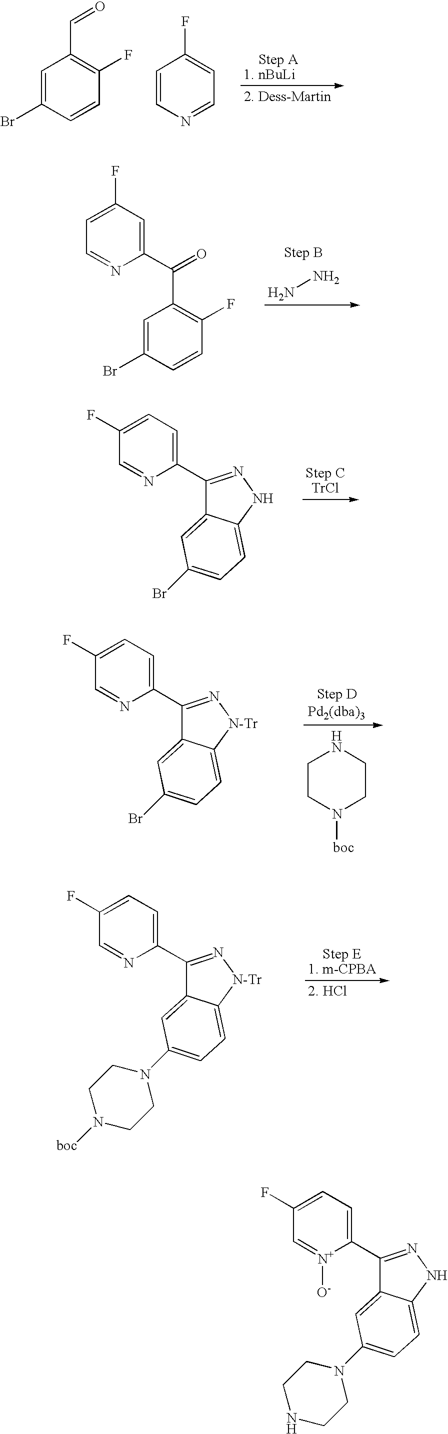

P-P2: For step F the protocol is described in JACS 2003, 125, 2890-2891

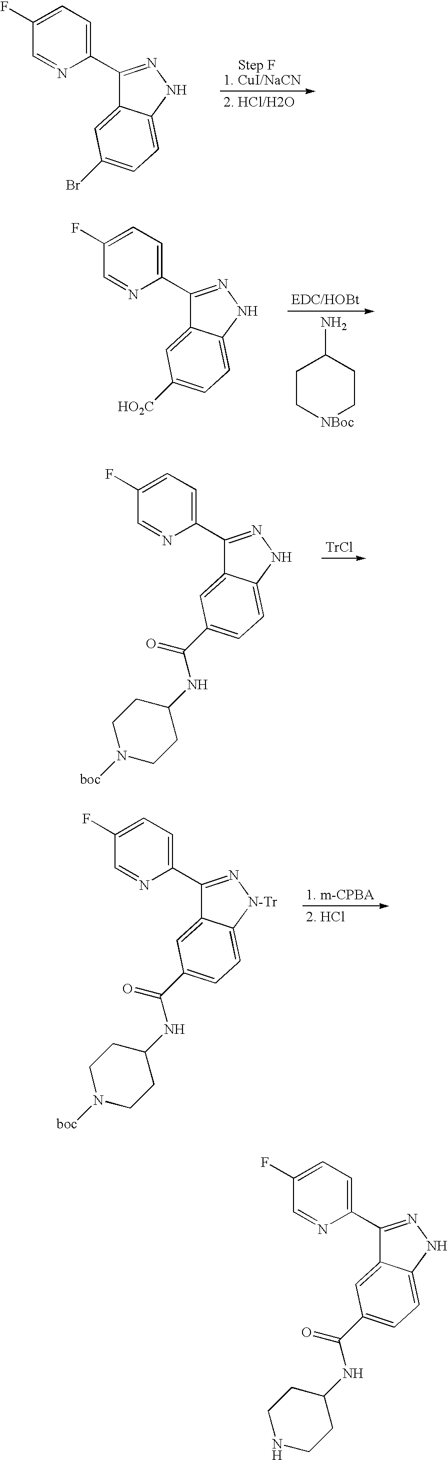

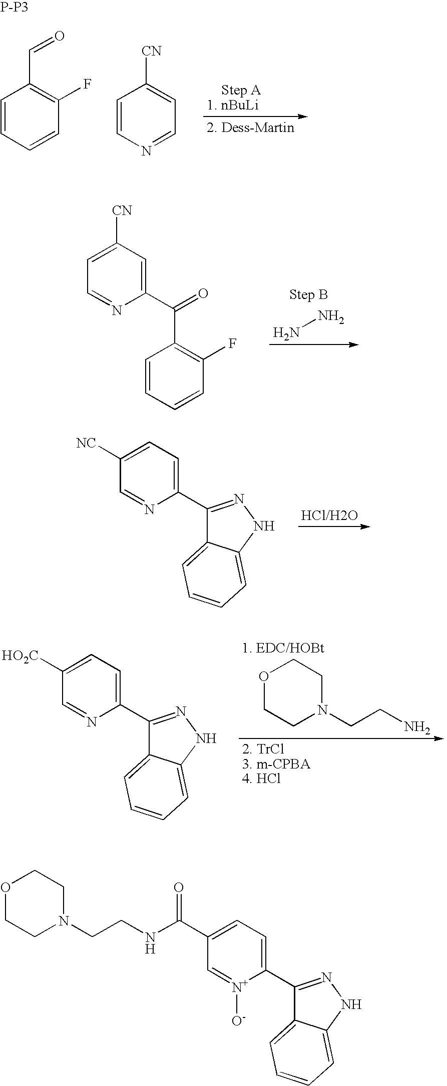

P-P4: The protocol for steps G and H are described in J. Med. Chem., 1992, 35, 3115-3129.

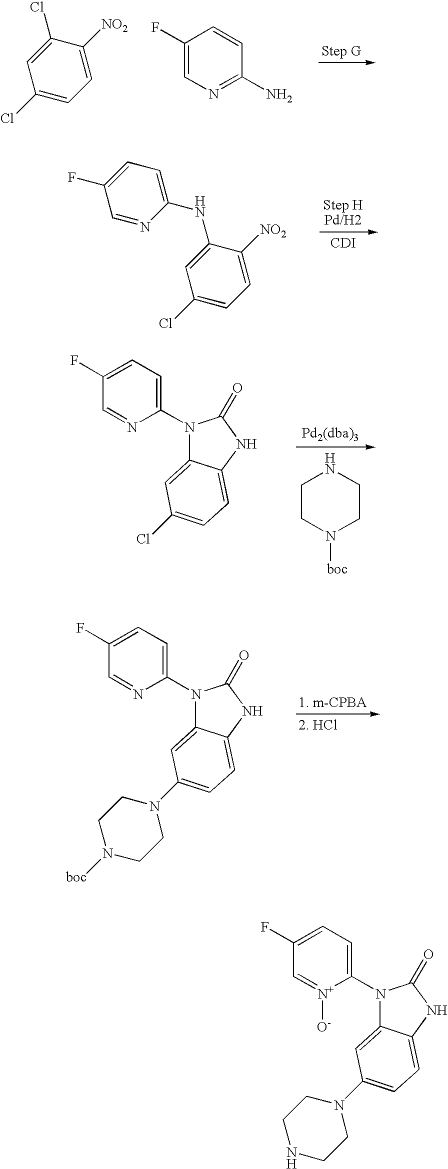

P-P5: 4-Chlorocarbonylpiperidine-1-carboxylic acid tert-butyl ester is documented in WO2000039117.

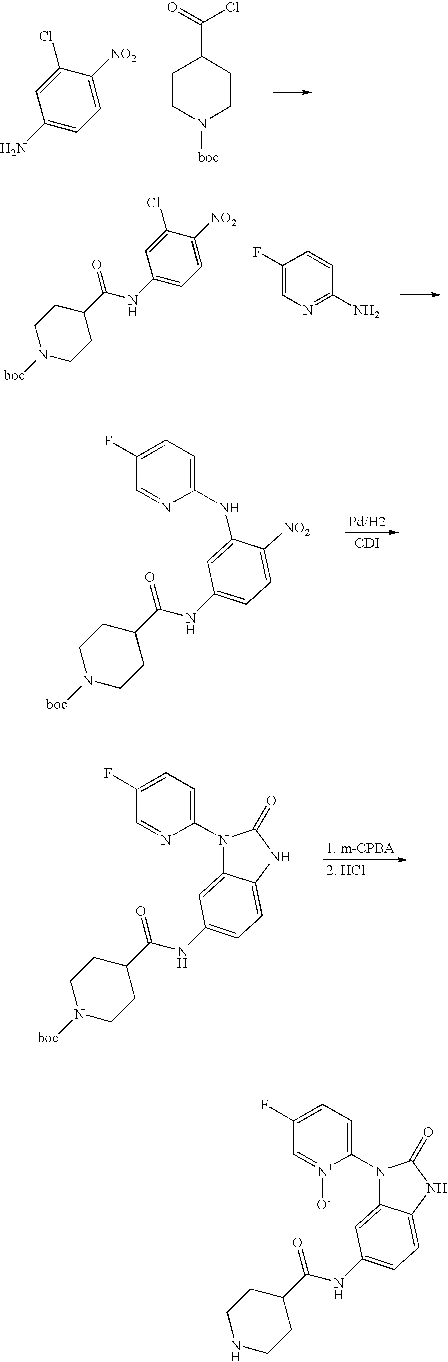

### Therapeutic Options for the Invention

The term “prodrug” refers to a metabolic precursor of a compound, and the term “active metabolite” refers to a metabolic product of a compound that is pharmaceutically acceptable and effective. As used herein, the unreduced mono-N-oxides of the Formulas I-III are referred to as prodrugs, and the reduced forms of these compounds exhibiting kinase inhibition are referred to as active metabolites. In accordance with the teachings provided herein, prodrugs and active metabolites of compounds of the Formulas I-III may be determined using techniques known in the art.

Prodrugs and active metabolites of a compound may be identified using routine techniques known in the art. See, e.g., Bertolini et al., J. Med. Chem., 40, 2011-2016 (1997); Shan, et al., J. Pharm. Sci., 86 (7), 765-767; Bagshawe, Drug Dev. Res., 34, 220-230 (1995); Bodor, Advances in Drug Res., 13, 224-331 (1984); Bundgaard, Design of Prodrugs (Elsevier Press 1985); and Larsen, Design and Application of Prodrugs, Drug Design and Development (Krogsgaard-Larsen et al., eds., Harwood Academic Publishers, 1991).

“A pharmaceutically acceptable salt” is intended to mean a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, y-hydroxybutyrates, glycollates, tartrates, methane-sulfonates, propanesulfonates, naphthalene-1-sulfonates, naphthalene-2-sulfonates, and mandelates.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the inventive compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In the case of agents that are solids, it is understood by those skilled in the art that the inventive compounds and salts may exist in different crystal or polymorphic forms, all of which are intended to be within the scope of the present invention and specified formulas.

The disclosed compounds of formula (I) and (Ia-e) and their pharmaceutically acceptable salts (referred to herein as “the present compounds”) are advantageously administered to inhibit protein kinases in a subject in whom a beneficial therapeutic or prophylactic effect can be achieved by inhibiting protein kinases, i.e., a subject in need of protein kinase inhibition. A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

The present compounds can be used to achieve a beneficial therapeutic or prophylactic effect, for example, in subjects with cancer. Cancers which can be treated with the present compounds include solid tumours such as colon, breast, lung, ovarian, pancreatic or non-solid tumours such as non-Hodgkins lymphomas and leukemias

The present compounds are also effective when used in combination with DNA-damaging anti-cancer drugs and/or radiation therapy to treat subjects with multi-drug resistant cancers. A cancer is resistant to a drug when it resumes a normal rate of tumour growth while undergoing treatment with the drug after the tumour had initially responded to the drug. A tumour “responds to a drug” when it exhibits a decrease in tumour mass or a decrease in the rate of tumour growth. The term “multi-drug resistant cancer” refers to cancer that is resistant to two or more drugs, typically five or more.

A pharmaceutical composition or preparation according to the invention comprises an effective amount of a protein-kinase modulating agent prodrug, optionally one or more other active agents, and optionally a pharmaceutically acceptable carrier, such as a diluent or excipient for the agent; when the carrier serves as a diluent, it may be a solid, semi-solid, or liquid material acting as a vehicle, excipient, or medium for the active ingredient(s). Compositions according to the invention may be made by admixing the active ingredient(s) with a carrier, or diluting it with a carrier, or enclosing or encapsulating it within a carrier, which may be in the form of a capsule, sachet, paper container, or the like. Exemplary ingredients, in addition to one or more protein kinase modulating agents and any other active ingredients, include Avicel (microcrystalline cellulose), starch, lactose, calcium sulfate dihydrate, terra alba, sucrose, talc, gelatin, agar, pectin, acacia, magnesium stearate, stearic acid, peanut oil, olive oil, glyceryl monostearate, Tween 80 (polysorbate 80), 1,3-butanediol, cocoa butter, beeswax, polyethylene glycol, propylene glycol, sorbitan monostearate, polysorbate 60, 2-octyldodecanol, benzyl alcohol, glycine, sorbic acid, potassium sorbate, disodium hydrogen phosphate, sodium chloride, and water.

The compositions may be prepared in any of a variety of forms suitable for the desired mode of administration. For example, pharmaceutical compositions may be prepared in the form of tablets, pills, powders, lozenges, sachets, cachets, elixirs, suspensions, emulsions, solutions, syrups, aerosols (as solids or in liquid media), ointments (e.g., containing up to 10% by weight of a protein kinase modulating agent), soft-gel and hard-gel capsules, suppositories, sterile injectable solutions, sterile packaged powders, and the like.

Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like.

A variety of pharmaceutical forms can be employed. Thus, if a solid carrier is used, the preparation can be tableted, placed in a hard gelatin capsule in powder or pellet form or in the form of a troche or lozenge. The amount of solid carrier may vary, but generally will be from about 25 mg to about 1 g. If a liquid carrier is used, the preparation can be in the form of syrup, emulsion, soft gelatin capsule, sterile injectable solution or suspension in an ampoule or vial or non-aqueous liquid suspension.

To obtain a stable water-soluble dose form, a pharmaceutically acceptable salt of an inventive agent is dissolved in an aqueous solution of an organic or inorganic acid, such as 0.3M solution of succinic acid or citric acid. If a soluble salt form is not available, the agent may be dissolved in a suitable cosolvent or combinations of cosolvents. Examples of suitable cosolvents include, but are not limited to, alcohol, propylene glycol, polyethylene glycol 300, polysorbate 80, glycerin and the like in concentrations ranging from 0-60% of the total volume. A compound of Formula I or I(a-e) may be dissolved in DMSO and diluted with water. The composition may also be in the form of a solution of a salt form of the active ingredient in an appropriate aqueous vehicle such as water or isotonic saline or dextrose solution.

Therapeutically effective amounts of the agents of the invention may be used to treat diseases mediated by modulation or regulation of protein kinases. An “effective amount” is intended to mean that amount of an agent that, when administered to a mammal in need of such treatment, is sufficient to effect treatment for a disease mediated by the activity of one or more kinases. Thus, e.g., a therapeutically effective amount of a compound of the Formula I or I(a-e), salt, active metabolite or prodrug thereof is a quantity sufficient to modulate, regulate, or inhibit the activity of one or more kinases such that a disease condition which is mediated by that activity is reduced or alleviated.

“Treating” is intended to mean at least the mitigation of a disease condition in a mammal, such as a human, that is affected, at least in part, by the activity of one or more kinases, and includes: preventing the disease condition from occurring in a mammal, particularly when the mammal is found to be predisposed to having the disease condition but has not yet been diagnosed as having it; modulating and/or inhibiting the disease condition; and/or alleviating the disease condition.

The amount of the present compounds administered to the subject will depend on the type and severity of the disease or condition and on the characteristics of the subject, such as general health, age, sex, body weight and tolerance to drugs. The skilled artisan will be able to determine appropriate dosages depending on these and other factors. Effective dosages for commonly used anti-cancer drugs and radiation therapy are well known to the skilled person. Effective amounts of the present compounds typically range between about 1 mg/m² per day and about 10 grams/m² per day, and preferably between 10 mg/m² per day and about 5 grams/m².

Techniques for formulation and administration of the compounds of the instant invention can be found in Remington: the Science and Practice of pharmacy, 19^{th} edition, Mack Publishing Co., Easton, Pa. (1995). The compositions of the invention may be manufactured in manners generally known for preparing pharmaceutical compositions, e.g., using conventional techniques such as mixing, dissolving, granulating, dragee-making, levigating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations which can be used pharmaceutically.

Proper formulation is dependent upon the route of administration chosen. For injection, the agents of the invention may be formulated into aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

For administration to the eye, the active agent is delivered in a pharmaceutically acceptable ophthalmic vehicle such that the compound is maintained in contact with the ocular surface for a sufficient time period to allow the compound to penetrate the corneal and internal regions of the eye, including, for example, the anterior chamber, posterior chamber, vitreous body, aqueous humor, vitreous humor, cornea, iris/ciliary, lens, choroid/retina and sclera. The pharmaceutically acceptable ophthalmic vehicle may be an ointment, vegetable oil, or an encapsulating material. A compound of the invention may also be injected directly into the vitreous and aqueous humor.

Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides.

The compounds may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

A pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) contains VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

Some of the compounds of the invention may be provided as salts with pharmaceutically compatible counter ions. Pharmaceutically compatible salts may be formed with many acids, including hydrochloric, sulfinuric, acetic, lactic, tartaric, malic, succinic, etc. Salts tend to be more soluble in aqueous or other protonic solvents than are the corresponding free-base forms.

A pharmaceutical composition according to the invention comprises a protein kinase modulating agent and, optionally, one or more other active ingredients, such as a known antiproliferative agent that is compatible with the protein kinase modulating agent and suitable for the indication being treated.

Preferably disclosed compounds or pharmaceutical formulations containing these compounds are in unit dosage form for administration to a mammal. The unit dosage form can be any unit dosage form known in the art including, for example, a capsule, an IV bag, a tablet, or a vial. The quantity of active ingredient (viz., a compound of Structural Formula I or I(a-e) or salts thereof) in a unit dose of composition is an effective amount and may be varied according to the particular treatment involved. It may be appreciated that it may be necessary to make routine variations to the dosage depending on the age and condition of the patient. The dosage will also depend on the route of administration which may be by a variety of routes including oral, aerosol, rectal, transdermal, subcutaneous, intravenous, intramuscular, intraperitoneal and intranasal.

1. A method for inhibiting the proliferation of cancer cells in a mammal, comprising administering to said mammal a therapeutically effective amount of a mono-N-oxide prodrug compound selected from the group consisting of compounds of the formulas I, II and III: 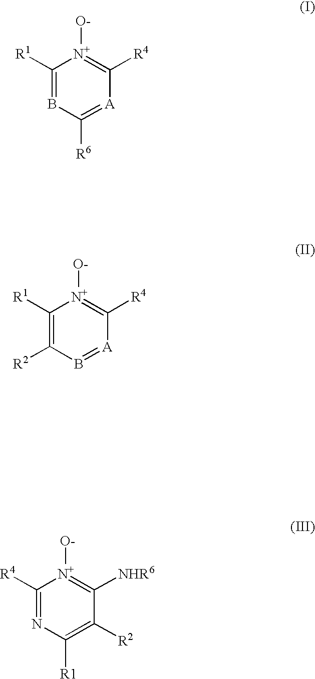
Wherein:
R¹ and R² are each independently selected from hydrogen, C₁-C₆ alkyl which is unsubstituted or substituted, C₁-C₆ haloalkyl, C₃-C₁₀ cycloalkyl which is unsubstituted or substituted, aryl which is unsubstituted or substituted, a 5- to 7-membered heterocyclic ring which is saturated or unsaturated and which may contain one or more heteroatoms selected from O, N and S and which is unsubstituted or substituted on any ring carbon or ring heteroatom, C₁-C₆ alkoxy which is unsubstituted or substituted, C₃-C₁₀ cycloalkoxy which is unsubstituted or substituted, halogen, hydroxyl, —OR⁶, —SR⁶, —SO₂R⁶, —SO₂N(R⁶)₂, —SO₂N(R⁷)(R⁸), —N(R⁶)₂, —N(R⁷)(R⁸), cyano, nitro, —COOR⁶, —C(O)N(R⁶)₂, —C(O)N(R⁷)(R⁸), —N(R⁶)C(O)R⁶, —N(R⁶)COOR⁶, —N(R⁶)CON(R⁶)₂, —N(R⁶)CON(R⁹)(R⁸), —N(R⁶)SO(R⁶), —N(R⁶)SO₂(R⁶), —C(O)R⁶, —OCH₂(CH₂)ₚN(R⁶)₂, —OCH₂(CH₂)ₚN(R⁷)(R⁸), —CH₂(CH₂)ₚN(R⁶)₂, —CH₂(CH₂)ₚN(R⁷)(R⁸), C(O)NHCH₂(CH₂)ₚN(R⁶)₂, —C(O)NHCH₂(CH₂)ₚN(R⁷)(R⁸), —NH(CH₂)ₚN(R⁶)₂, —NH(CH₂)ₚN(R⁷)(R⁸), —NHC(O)CH₂(CH₂)ₚN(R⁷)(R⁸), —NHC(O)CH₂(CH₂)ₚN(R⁶)₂, —(CH₂)_{q}C(O)R⁶, —OCH₂CH₂OR⁶, —O(CH₂)_{q}C(O)R⁶, —O(CH₂)_{q}(OCH₂CH₂)_{q}OR⁶, XN(R⁶)₂ or —X—N(R⁷)(R⁸)
wherein X is a C₁-C₆ alkylidine group that is optionally interrupted by —O—, —S—, —C(O)— or —N(R⁶), and wherein
R¹ and R² may form, together with the carbon atoms to which they are attached, a fused benzene ring or a fused 5- to 7-membered heterocyclic ring which is saturated or unsaturated and which may contain one or more heteroatoms selected from O, N, and S, the benzene ring or heterocyclic ring being unsubstituted or substituted;
wherein R⁶ is H, C₁-C₆ alkyl which is unsubstituted or substituted, C₃-C₁₀ cycloalkyl which is unsubstituted or substituted, a 5- to 7-membered heterocyclic ring which is unsaturated or saturated and which contains one or more heteroatoms selected from O, N and S and which is unsubstituted or substituted on any ring carbon or ring heteroatom, an aromatic or heteroaromatic ring optionally substituted by halogen, hydroxyl, —OR¹⁰, —SR¹⁰, —SO₂R¹⁰, —SO₂N(R¹⁰)₂, —N(R¹⁰)₂, —N(R⁷)(R⁸), cyano, nitro, —COOR¹⁰, —C(O)N(R¹⁰)₂, —N(R¹⁰)C(O)R¹⁰, —N(R¹⁰)COOR¹⁰, —N(R¹⁰)CON(R¹⁰)₂, —N(R¹⁰)SO(R¹⁰), —N(R¹⁰)SO₂(R¹⁰), —C(O)R¹⁰ and aromatic or heteroaromatic ring optionally substituted by two R¹⁰ that may be taken together to form a fused bicyclic system, and wherein more than one R⁶ attached to the same nitrogen atom is the same or different;
R⁷ and R⁸ form, together with the N atom to which they are attached, a 3- to 9-membered N-containing heterocyclic ring which is unsaturated or saturated and which may contain one or more heteroatoms selected from O, N and S and which is unsubstituted or substituted on any ring carbon or ring heteroatom;
p is 0 or an integer from 1 to 5;
q is an integer from 1 to 6;
A and B are optionally and independently N or CR³
wherein R³ is optionally H, NHR⁶, OR⁶, SR⁶, or selected from C₁-C₆ alkyl which is unsubstituted or substituted, C₁-C₆ alkoxy which is unsubstituted or substituted, C₃-C₁₀ cycloalkoxy which is unsubstituted or substituted, phenyl which is unsubstituted or substituted, halogen, hydroxyl, SOR⁶, SO₂R⁶, SONHR⁶, NO₂, cyano, N(R⁶)₂, NHCON(R⁶)₂ or NHCON(R⁷)(R⁸), COOR₆, NR⁷R⁸ wherein each R⁶ is the same or different and wherein R³ groups on adjacent carbon atoms can, together with the carbon atoms to which they are attached, form an aromatic ring which may be substituted with one or more R⁶ groups;
R⁴ is optionally H, NHR⁶, SR⁶, C₁-C₆ alkyl which is unsubstituted or substituted and which is optionally interrupted by —O—, —S—, —C(O)— or —N(R⁶)—, C₃-C₈ cycloalkyl which is unsubstituted or substituted, aryl which is unsubstituted or substituted or a 5- to 7-membered heterocyclic group which is unsaturated or saturated, which contains 1 or more heteroatoms selected from O, N and S and which is unsubstituted or substituted on any ring carbon or ring heteroatom, or R⁴ and A, together with the C atoms to which they are attached, form a 5-membered N-containing heterocyclic ring, which is saturated or unsaturated and which may contain one or more heteroatoms selected from O, N and S and which is unsubstituted or substituted on any ring carbon or ring heteroatom; and
R¹⁰ is H or C₁-C₆ alkyl which is unsubstituted or substituted, C₃-C₁₀ cycloalkyl which is unsubstituted or substituted or a 5- to 7-membered heterocyclic ring which is unsaturated or saturated which contains one or more heteroatoms selected from O, N and S and which is unsubstituted or substituted on any ring carbon or ring heteroatom and wherein more than one R¹⁰ attached to the same nitrogen atom is the same or different;
or a pharmaceutically acceptable salt of a compound of the Formulas (I), (II) or (III).

2. The method of claim 1, wherein said prodrug compound is selectively reduced to a therapeutically active metabolite in a hypoxic environment.

3. The method of claim 2, wherein said mono-N-oxide moiety has a one electron reduction potential less than −300 mV.

4. The method of claim 2, wherein said mono-N-oxide moiety has a one electron reduction potential in the range of from about −400 mV to about −510 mV.

5. The method of claim 4, further comprising the administration of ionizing radiation to said cancer cells.

6. The method of claim 4, further comprising the administration of a chemotherapeutic agent that imparts oxidative damage to DNA of said cancer cells.

7. The method of claim 6, wherein said chemotherapeutic agent is Tirapazamine.

8. The method of claim 5, wherein said prodrug administration enhances the cytotoxicity of said ionizing radiation in hypoxic tumor cells.

9. The method of claim 7, wherein said prodrug administration enhances the cytotoxicity of said Tirapazamine in hypoxic tumor cells.

10. The method of claim 2, wherein said active metabolite inhibits the activity of protein kinases within the cancer cells.

11. The method of claim 10, wherein said protein kinases are selected from the group consisting of ab1, Arg, KDR, Flt-1, c-Kit, c-Raf, cSRC, FGFR1, JNK1+1, MAPK2, MEK1, EGFR, ERBBZ, PDGFR, cMet, TIEZ, RET, VEGFR, IGF-1R, Akt, P70S6, PKA, PKC, PI3K, PDK1, PDK2, Cdk1, Cdk2, Cdk4, Myt1, Chk1, Wee1, AuroraA, AuroraB, Plk, Bulb1, Bulb3, Chk2, ATM, ATR, CKII, and DNA-PK.

12. The method of claim 10, wherein said protein kinases are selected from the group consisting of AuroraA, Chk1, KDR, VEGFR, P70S6K, ab1, ARG, and CK2.

13. The method of claim 1, wherein said mono-N-oxide prodrug is a compound of the formula II(a): 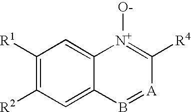
wherein,
R¹ and R² are each independently selected from hydrogen, C₁-C₆ alkyl which is unsubstituted or substituted, C₁-C₆ haloalkyl, C₃-C₁₀ cycloalkyl which is unsubstituted or substituted, aryl which is unsubstituted or substituted, a 5- to 7-membered heterocyclic ring which is saturated or unsaturated and which may contain one or more heteroatoms selected from O, N and S and which is unsubstituted or substituted on any ring carbon or ring heteroatom, C₁-C₆ alkoxy which is unsubstituted or substituted, C₃-C₁₀ cycloalkoxy which is unsubstituted or substituted, halogen, hydroxyl, —OR⁶, —SR⁶, —SO₂R⁶, —SO₂N(R⁶)₂, —SO₂N(R⁷)(R⁸), —N(R⁶)₂, —N(R⁷)(R⁸), cyano, nitro, —COOR⁶, —C(O)N(R⁶)₂, —C(O)N(R⁷)(R⁸), —N(R⁶)C(O)R⁶, —N(R⁶)COOR⁶, —N(R⁶)CON(R⁶)₂, —N(R⁶)CON(R⁹)(R⁸), —N(R⁶)SO(R⁶), —N(R⁶)SO₂(R⁶), —C(O)R⁶, —OCH₂(CH₂)ₚN(R⁶)₂, —OCH₂(CH₂)ₚN(R⁷)(R⁸), —CH₂(CH₂)ₚN(R⁶)₂, —CH₂(CH₂)ₚN(R⁷)(R⁸), C(O)NHCH₂(CH₂)ₚN(R⁶)₂, —C(O)NHCH₂(CH₂)ₚN(R⁷)(R⁸), —NH(CH₂)ₚN(R⁶)₂, —NH(CH₂)ₚN(R⁷)(R⁸), —NHC(O)CH₂(CH₂)ₚN(R⁷)(R⁸), —NHC(O)CH₂(CH₂)ₚN(R⁶)₂, —(CH₂)_{q}C(O)R⁶, —OCH₂CH₂OR⁶, —O(CH₂)_{q}C(O)R⁶, —O(CH₂)_{q}(OCH₂CH₂)_{q}OR⁶, XN(R⁶)₂ or —X—N(R⁷)(R⁸)
wherein X is a C₁-C₆ alkylidine group that is optionally interrupted by —O—, —S—, —C(O)— or —N(R⁶), and wherein
wherein R⁶ is H, C₁-C₆ alkyl which is unsubstituted or substituted, C₃-C₁₀ cycloalkyl which is unsubstituted or substituted, a 5- to 7-membered heterocyclic ring which is unsaturated or saturated and which contains one or more heteroatoms selected from O, N and S and which is unsubstituted or substituted on any ring carbon or ring heteroatom, an aromatic or heteroaromatic ring optionally substituted by halogen, hydroxyl, —OR¹⁰, —SR¹⁰, —SO₂R¹⁰, —SO₂N(R¹⁰)₂, —N(R¹⁰)₂, —N(R⁷)(R⁸), cyano, nitro, —COOR¹⁰, —C(O)N(R¹⁰)₂, —N(R¹⁰)C(O)R¹⁰, —N(R¹⁰)COOR¹⁰, —N(R¹⁰)CON(R¹⁰)₂, —N(R¹⁰)SO(R¹⁰), —N(R¹⁰)SO₂(R¹⁰), —C(O)R¹⁰ and aromatic or heteroaromatic ring optionally substituted by two R¹⁰ that may be taken together to form a fused bicyclic system, and wherein more than one R⁶ attached to the same nitrogen atom is the same or different;
R⁷ and R⁸ form, together with the N atom to which they are attached, a 3- to 9-membered N-containing heterocyclic ring which is unsaturated or saturated and which may contain one or more heteroatoms selected from O, N and S and which is unsubstituted or substituted on any ring carbon or ring heteroatom;
p is 0 or an integer from 1 to 5;
q is an integer from 1 to 6;
A and B are optionally and independently N or CR³
wherein R³ is optionally H, NHR⁶, OR⁶, SR⁶, or selected from C₁-C₆ alkyl which is unsubstituted or substituted, C₁-C₆ alkoxy which is unsubstituted or substituted, C₃-C₁₀ cycloalkoxy which is unsubstituted or substituted, phenyl which is unsubstituted or substituted, halogen, hydroxyl, SOR⁶, SO₂R⁶, SONHR⁶, NO₂, cyano, N(R⁶)₂, NHCON(R⁶)₂ or NHCON(R⁷)(R⁸), COOR₆, NR⁷R⁸ wherein each R⁶ is the same or different and wherein R³ groups on adjacent carbon atoms can, together with the carbon atoms to which they are attached, form an aromatic ring which may be substituted with one or more R⁶ groups;
R⁴ is optionally H, NHR⁶, SR⁶, C₁-C₆ alkyl which is unsubstituted or substituted and which is optionally interrupted by —O—, —S—, —C(O)— or —N(R⁶)—, C₃-C₈ cycloalkyl which is unsubstituted or substituted, aryl which is unsubstituted or substituted or a 5- to 7-membered heterocyclic group which is unsaturated or saturated, which contains 1 or more heteroatoms selected from O, N and S and which is unsubstituted or substituted on any ring carbon or ring heteroatom, or R⁴ and A, together with the C atoms to which they are attached, form a 5-membered N-containing heterocyclic ring, which is saturated or unsaturated and which may contain one or more heteroatoms selected from O, N and S and which is unsubstituted or substituted on any ring carbon or ring heteroatom; and
R¹⁰ is H or C₁-C₆ alkyl which is unsubstituted or substituted, C₃-C₁₀ cycloalkyl which is unsubstituted or substituted or a 5- to 7-membered heterocyclic ring which is unsaturated or saturated which contains one or more heteroatoms selected from O, N and S and which is unsubstituted or substituted on any ring carbon or ring heteroatom and wherein more than one R¹⁰ attached to the same nitrogen atom is the same or different;
or a pharmaceutically acceptable salt thereof.

14. The method of claim 13, wherein said mono-N-oxide prodrug is a compound of the formula II(b): 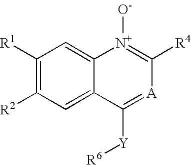
wherein Y is O, S, NH, N(R⁷)(R⁸); or a pharmaceutically acceptable salt thereof.

15. The method of claim 13, wherein said mono-N-oxide prodrug is a compound of the formula II(c): 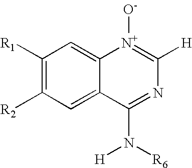
or a pharmaceutically acceptable salt thereof.

16. The method of claim 13, wherein said mono-N-oxide prodrug is a compound of the formula II(d): 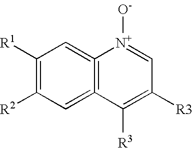
or a pharmaceutically acceptable salt thereof.

17. The method of claim 13, wherein said mono-N-oxide prodrug is a compound of the formula II(e): 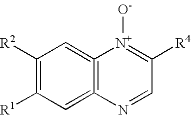
or a pharmaceutically acceptable salt thereof.

18. The method of claim 1, wherein said mono-N-oxide prodrug is a compound of the formula II(f): 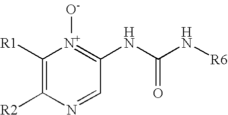
or a pharmaceutically acceptable salt thereof.

19. The method of claim 1, wherein said mono-N-oxide prodrug is a compound of the formula II(g): 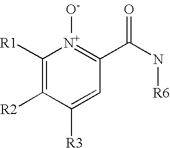
or a pharmaceutically acceptable salt thereof.

20. The method of claim 1, wherein said mono-N-oxide prodrug is a compound of the formula II(h): 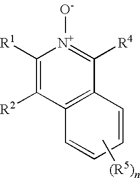
Wherein each R⁵, which are the same or different, are as described for R¹ and R²; and n is an integer from 1 to 4;
or a pharmaceutically acceptable salt thereof.

21. The method of claim 1, wherein said mono-N-oxide prodrug is a compound of the formula II(i): 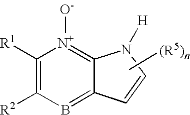
wherein R⁵ is as described for R¹ and R²; and N is 1 or 2; or a pharmaceutically acceptable salt thereof.

22. The method of claim 1, wherein said mono-N-oxide prodrug is a compound of the formula I(a): 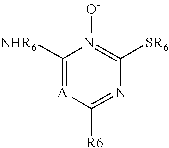
or a pharmaceutically acceptable salt thereof.

23. A product for the selective inhibition of hypoxic cancer cell proliferation, selected from the group consisting of compounds of the formulas I, II and III: 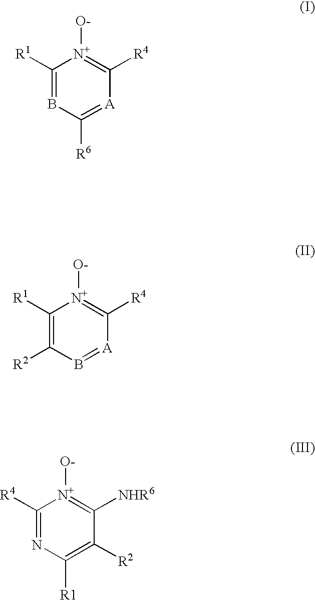
Wherein:
R¹ and R² are each independently selected from hydrogen, C₁-C₆ alkyl which is unsubstituted or substituted, C₁-C₆ haloalkyl, C₃-C₁₀ cycloalkyl which is unsubstituted or substituted, aryl which is unsubstituted or substituted, a 5- to 7-membered heterocyclic ring which is saturated or unsaturated and which may contain one or more heteroatoms selected from O, N and S and which is unsubstituted or substituted on any ring carbon or ring heteroatom, C₁-C₆ alkoxy which is unsubstituted or substituted, C₃-C₁₀ cycloalkoxy which is unsubstituted or substituted, halogen, hydroxyl, —OR⁶, —SR⁶, —SO₂R⁶, SO₂N(R⁶)₂, —SO₂N(R⁷)(R⁸), —N(R⁶)₂, —N(R⁷)(R⁸), cyano, nitro, —COOR⁶, —C(O)N(R⁶)₂, —C(O)N(R⁷)(R⁸), —N(R⁶)C(O)R⁶, —N(R⁶)COOR⁶, —N(R⁶)CON(R⁶)₂, —N(R⁶)CON(R⁹)(R⁸), —N(R⁶)SO(R⁶), —N(R⁶)SO₂(R⁶), —C(O)R⁶, —OCH₂(CH₂)ₚN(R⁶)₂, —OCH₂(CH₂)ₚN(R⁷)(R⁸), —CH₂(CH₂)ₚN(R⁶)₂, —CH₂(CH₂)ₚN(R⁷)(R⁸), C(O)NHCH₂(CH₂)ₚN(R⁶)₂, —C(O)NHCH₂(CH₂)ₚN(R⁷)(R⁸), —NH(CH₂)ₚN(R⁶)₂, —NH(CH₂)ₚN(R⁷)(R⁸), —NHC(O)CH₂(CH₂)ₚN(R⁷)(R⁸), —NHC(O)CH₂(CH₂)ₚN(R⁶)₂, —(CH₂)_{q}C(O)R⁶, —OCH₂CH₂OR⁶, —O(CH₂)_{q}C(O)R⁶, —O(CH₂)_{q}(OCH₂CH₂)_{q}OR⁶, XN(R⁶)₂ or —X—N(R⁷)(R⁸)
wherein X is a C₁-C₆ alkylidine group that is optionally interrupted by —O—, —S—, —C(O)— or —N(R⁶), and wherein
R¹ and R² may form, together with the carbon atoms to which they are attached, a fused benzene ring or a fused 5- to 7-membered heterocyclic ring which is saturated or unsaturated and which may contain one or more heteroatoms selected from O, N, and S, the benzene ring or heterocyclic ring being unsubstituted or substituted;
wherein R⁶ is H, C₁-C₆ alkyl which is unsubstituted or substituted, C₃-C₁₀ cycloalkyl which is unsubstituted or substituted, a 5- to 7-membered heterocyclic ring which is unsaturated or saturated and which contains one or more heteroatoms selected from O, N and S and which is unsubstituted or substituted on any ring carbon or ring heteroatom, an aromatic or heteroaromatic ring optionally substituted by halogen, hydroxyl, —OR¹⁰, —SR¹⁰, —SO₂R¹⁰, —SO₂N(R¹⁰)₂, —N(R¹⁰)₂, —N(R⁷)(R⁸), cyano, nitro, —COOR¹⁰, —C(O)N(R¹⁰)₂, —N(R¹⁰)C(O)R¹⁰, —N(R¹⁰)COOR¹⁰, —N(R¹⁰)CON(R¹⁰)₂, —N(R¹⁰)SO(R¹⁰), —N(R¹⁰)SO₂(R¹⁰), —C(O)R¹⁰ and aromatic or heteroaromatic ring optionally substituted by two R¹⁰ that may be taken together to form a fused bicyclic system, and wherein more than one R⁶ attached to the same nitrogen atom is the same or different.
R⁷ and R⁸ form, together with the N atom to which they are attached, a 3- to 9-membered N-containing heterocyclic ring which is unsaturated or saturated and which may contain one or more heteroatoms selected from O, N and S and which is unsubstituted or substituted on any ring carbon or ring heteroatom;
p is 0 or an integer from 1 to 5;
q is an integer from 1 to 6;
A and B are optionally and independently N or CR³
wherein R³ is optionally H, NHR⁶, OR⁶, SR⁶, or selected from C₁-C₆ alkyl which is unsubstituted or substituted, C₁-C₆ alkoxy which is unsubstituted or substituted, C₃-C₁₀ cycloalkoxy which is unsubstituted or substituted, phenyl which is unsubstituted or substituted, halogen, hydroxyl, SOR⁶, SO₂R⁶, SONHR⁶, NO₂, cyano, N(R⁶)₂, NHCON(R⁶)₂ or NHCON(R⁷)(R⁸), COOR₆, NR⁷R⁸ wherein each R⁶ is the same or different and wherein R³ groups on adjacent carbon atoms can, together with the carbon atoms to which they are attached, form an aromatic ring which may be substituted with one or more R⁶ groups;
R⁴ is optionally H, NHR⁶, SR⁶, C₁-C₆ alkyl which is unsubstituted or substituted and which is optionally interrupted by —O—, —S—, —C(O)— or —N(R⁶)—, C₃-C₈ cycloalkyl which is unsubstituted or substituted, aryl which is unsubstituted or substituted or a 5- to 7-membered heterocyclic group which is unsaturated or saturated, which contains 1 or more heteroatoms selected from O, N and S and which is unsubstituted or substituted on any ring carbon or ring heteroatom, or R⁴ and A, together with the C atoms to which they are attached, form a 5-membered N-containing heterocyclic ring, which is saturated or unsaturated and which may contain one or more heteroatoms selected from O, N and S and which is unsubstituted or substituted on any ring carbon or ring heteroatom; and
R¹⁰ is H or C₁-C₆ alkyl which is unsubstituted or substituted, C₃-C₁₀ cycloalkyl which is unsubstituted or substituted or a 5- to 7-membered heterocyclic ring which is unsaturated or saturated which contains one or more heteroatoms selected from O, N and S and which is unsubstituted or substituted on any ring carbon or ring heteroatom and wherein more than one R¹⁰ attached to the same nitrogen atom is the same or different;
or a pharmaceutically acceptable salt of a compound of the Formula I.

24. The prodrug of claim 23, wherein the mono-N-oxide moiety has a one electron reduction potential less than −300 mV.

25. The prodrug of claim 23, wherein said mono-N-oxide moiety has a one electron reduction potential in the range of from about −400 mV to about −510 mV.

26. The prodrug of claim 23, wherein said mono-N-oxide moiety has a one electron reduction potential in the range of from about −450mV to about −510 mV.

27. The prodrug of claim 23, wherein said prodrug undergoes reduction of the mono-N-oxide moiety in a hypoxic environment to form a metabolite that inhibits protein kinase activity.

28. The prodrug of claim 27, which, upon reduction of the mono-N-oxide moiety, yield a metabolite that inhibits the activity of protein kinases selected from the group consisting of EGFR, ERBBZ, PDGFR, cMet, TIEZ, RET, VEGFR, IGF-1R, Akt, P70s6, PKA, PDK1, PDK2, Cdk1, Cdk2, Cdk4, Myt1, Chk1, Wee1, AuroraA, AuroraB, Plk, Bulb1, Bulb3, Chk2, ATM, ATR, CKII, and DNA-PK.

29. The prodrug of claim 27, which, upon reduction of the mono-N-oxide moiety, yield a metabolite that inhibits the activity of protein kinases selected from the group consisting of AuroraA, Chk1, KDR, VEGFR, P70S6K, ab1, ARG, and CKII.

30. The prodrug of claim 23, wherein said prodrug is a compound of the formula: 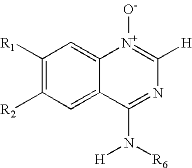
or a pharmaceutically acceptable salt thereof.

31. The prodrug of claim 23, wherein said prodrug is a compound of the formula: 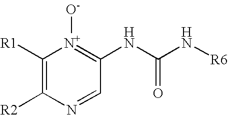
or a pharmaceutically acceptable salt thereof.

32. The prodrug of claim 23, wherein said prodrug is a compound of the formula: 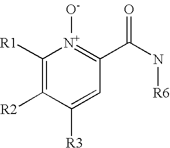
or a pharmaceutically acceptable salt thereof.

33. The prodrug of claim 23, wherein said prodrug is a compound of the formula: 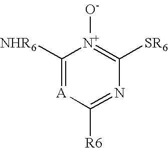
or a pharmaceutically acceptable salt thereof.

34. A pharmaceutical composition comprising the prodrug of claim 23.

35. A pharmaceutical composition comprising the prodrug of claim 29.

36. A pharmaceutical composition comprising the prodrug of claim 30.

37. A pharmaceutical composition comprising the prodrug of claim 31.

38. A pharmaceutical composition comprising the prodrug of claim 32.

39. A pharmaceutical composition comprising the prodrug of claim 33.
